(19) United States
(12) Patent Application Publication
(10) Pub. No.: US 2002/0064831 A1
(43) Pub. Date: May 30, 2002

(54) Nucleic acid sequences encoding CMG proteins, CMG proteins, and methods for their use

(75) Inventors: George E. Davis, College Station, TX (US); Scott E. Bell, Bryan, TX (US)

Correspondence Address: Christopher J. Buntel, Ph.D., Howrev Simon Arnold & White, LLP, 750 Bering Drive, Houston, TX, 77057-2198, US

(73) Assignee: The Texas A&M University System

(21) Appl. No.: 09/975,901

(22) Filed: Oct. 12, 2001

Related U.S. Application Data
(60) Provisional application No. 60/239,772, filed on Oct. 12, 2000.

(51) Int. Cl. C12N 9/64; C12N 15/861; C07H 21/04

(52) U.S. Cl. 435/69.1; 435/320.1; 435/325; 536/23.2; 435/456; 435/226

(57) ABSTRACT

Differential gene expression assays were used to identify a number of sequences in an in vitro model of human capillary tube formation. Nucleic acid sequences encoding the capillary morphogenesis gene CMG-1 and CMG-2 proteins are disclosed. The nucleic acids and proteins are useful in constructing vectors, recombinant cells, fusion proteins, and in methods for the isolation of matrix proteins.

### CROSS REFERENCE TO RELATED APPLICATIONS

The present application is related to U.S. Provisional Patent Application Serial No. 60/239,772 filed Oct. 12, 2000.

The government may own rights in the present invention pursuant to grant number 1RO1HL59373-01 from the National Institutes of Health.

### FIELD OF THE INVENTION

The invention relates to nucleic acids encoding capillary morphogenesis proteins, their encoded proteins, and methods for their use. In particular, the CMG-1 and CMG-2 nucleic acids, and their encoded proteins are disclosed.

### BACKGROUND OF THE INVENTION

Studies on the molecular control of endothelial cell (EC) morphogenesis during angiogenesis or vasculogenesis have revealed many new insights into how blood vessels participate in complex biological processes such as development, wound repair and tumorigenesis (Hanahan, 1997; Carmeliet and Jain, 2000; Yancopoulos et al., 2000; Conway et al., 2001). Although considerable work has been performed identifying factors which promote or inhibit angiogenic responses (Folkman, 1997; Carmeliet and Jain, 2000; Yancopoulos et al., 2000; Conway et al., 2001), considerably less effort has focused on how individual and groups of ECs assemble into capillary tubes during these events. Identifying new molecular targets that block specific steps in EC morphogenesis may become critical in efforts to inhibit angiogenesis in human diseases where angiogenesis is a pathogenic component (i.e. cancer, diabetic retinopathy, arthritis, atherosclerosis)(Folkman, 1995; Carmeliet and Jain, 2000).

One experimental approach to address these questions has been to utilize in vitro models of EC morphogenesis where many of the steps observed in vivo can be mimicked (Montesano et al., 1992; Vernon and Sage, 1995; Nicosia and Villaschi, 1999). The most promising assays for elucidating relevant molecules and pathways necessary for EC morphogenesis are those utilizing three-dimensional extracellular matrices (ECM) composed of collagen type I or fibrin (Montesano and Orci, 1985; Nicosia and Ottinetti, 1990; Davis and Camarillo, 1996; Ilan et al., 1998; Vernon and Sage, 1999; Yang et al., 1999; Bayless et al., 2000; Davis et al., 2000). These matrices represent the major matrix environments where angiogenic or vasculogenic events take place (Vernon and Sage, 1995; Senger, 1996; Nicosia and Villaschi, 1999). In some of these assays, particularly where ECs are suspended as individual cells in three-dimensional matrices, most of the ECs undergo morphogenesis simultaneously, which allows for an analysis of differential gene expression in large numbers of ECs. This is a critical aspect of EC morphogenic or regression microassays developed by our laboratory (Davis and Camarillo, 1996; Bayless et al., 2000; Davis et al., 2000; Davis et al., 2001). In these systems, differential gene expression can be directly correlated with distinct events in the EC morphogenic or regression cascade (Salazar et al., 1999; Davis et al., 2001).

Many studies over the years have shown that differential gene expression controls complex biological phenomena (Brown and Botstein, 1999). Recently, the development of gene array technology has revealed how classes of differentially regulated genes control processes such as yeast responses to glucose deprivation, fibroblast responses to serum mitogens and tumor development and apoptosis (DeRisi et al., 1997; Iyer et al., 1999; Perou et al., 2000; Maxwell and Davis, 2000). These approaches have been useful to characterize the role of previously identified genes in a given process. To identify relevant differentially expressed novel genes, additional techniques were developed including differential display, subtraction cDNA cloning and serial analysis of gene expression (SAGE) (Velculescu et al., 1995; Martin and Pardee, 1999). Using this latter technology, differentially regulated genes (known and novel) were identified in colon carcinoma-derived endothelium versus normal colonic endothelium (St. Croix et al., 2000).

Thus, there exists a need for new methods for the identification of differentially regulated sequences, and for the nucleic acid and protein sequences themselves.

### SUMMARY OF THE INVENTION

Nucleic acid sequences encoding the CMG-1 and CMG-2 proteins are disclosed, along with the deduced amino acid sequence of the proteins. Methods of their use in various analytical and preparative applications are also presented.

### DESCRIPTION OF THE SEQUENCE LISTINGS

The following sequence listings form part of the present specification and are included to further demonstrate certain aspects of the present invention. The invention may be better understood by reference to one or more of these sequences in combination with the detailed description of specific embodiments presented herein.

| SEQ ID NO: | Description |
|---|---|
| 1 | Nucleic acid sequence encoding CMG-1 protein |
| 2 | Amino acid sequence of CMG-1 protein |
| 3 | Nucleic acid sequence encoding CMG-2 protein |
| 4 | Amino acid sequence of CMG-2 protein |
| 5 | Amino acid sequence of CMG-2 protein residues 34-214 |
| 6 | CMG-1 primer 1 |
| 7 | CMG-1 primer 2 |
| 8 | CMG-2 primer 1 |
| 9 | CMG-2 primer 2 |
| 10 | Common downstream primer |
| 11 | CMG-1 upstream primer |
| 12 | CMG-2 upstream primer |
| 13 | CMG-2 amplification primer 1 |
| 14 | CMG-2 amplification primer 2 |
| 15 | CMG-1 complete isolated nucleic acid sequence |
| 16 | CMG-2 complete isolated nucleic acid sequence |
| 17 | CMG-2 Export peptide sequence |
| 18 | CMG-2 Integrin a subunit I-domain |
| 19 | CMG-2 vWF A-domain |
| 20 | CMG-2 transmembrane domain |
| 21 | CMG-2 WASP WH-1 domain |

### DEFINITIONS

The following definitions are provided in order to aid those skilled in the art in understanding the detailed description of the present invention.

Amino acid codes: A (Ala)=alanine; C (Cys)=cysteine; D (Asp)=aspartic acid; E (Glu)=glutamic acid; F (Phe)=phenylalanine; G (Gly)=glycine; H (His)=histidine; I (Ile)=isoleucine; K (Lys)=lysine; L (Leu)=leucine; M (Met)=methionine; N (Asn)=asparagine; P (Pro)=proline; Q (Gln)=glutamine; R (Arg)=arginine; S (Ser)=serine; T (Thr)=threonine; V (Val)=valine; W (Trp)=tryptophan; Y (Tyr)=tyrosine.

“Coding sequence”, “open reading frame”, and “structural sequence” refer to the region of continuous sequential nucleic acid triplets encoding a protein, polypeptide, or peptide sequence.

“Codon” refers to a sequence of three nucleotides that specify a particular amino acid.

“Expression” refers to the transcription of a gene to produce the corresponding mRNA and translation of this mRNA to produce the corresponding gene product, i.e., a peptide, polypeptide, or protein.

“Expression of antisense RNA” refers to the transcription of a DNA to produce an first RNA molecule capable of hybridizing to a second RNA molecule encoding a gene product, e.g. a protein. Formation of the RNA-RNA hybrid inhibits translation of the second RNA molecule to produce the gene product.

“Hybridization” refers to the ability of a strand of nucleic acid to join with a complementary strand via base pairing. Hybridization occurs when complementary sequences in the two nucleic acid strands bind to one another.

“Identity” refers to the degree of similarity between two nucleic acid or protein sequences. An alignment of the two sequences is performed by a suitable computer program. A widely used and accepted computer program for performing sequence alignments is CLUSTALW v1.6 (Thompson, et al. Nucl. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there were 100 matched amino acids between 200 and a 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

“Nucleic acid” refers to deoxyribonucleic acid (DNA) and ribonucleic acid (RNA).

Nucleic acid codes: A=adenosine; C=cytosine; G=guanosine; T=thymidine; N=equimolar A, C, G, and T; I=deoxyinosine; K=equimolar G and T; R=equimolar A and G; S=equimolar C and G; W=equimolar A and T; Y=equimolar C and T.

“Nucleic acid segment” refers to a nucleic acid molecule that has been isolated free of total genomic DNA of a particular species, or that has been synthesized. Included with the term “nucleic acid segment” are DNA segments, recombinant vectors, plasmids, cosmids, phagemids, phage, viruses, etcetera.

“Open reading frame (ORF)” refers to a region of DNA or RNA encoding a peptide, polypeptide, or protein.

### DETAILED DESCRIPTION OF THE INVENTION

CMG-1 Related Inventions

One embodiment of the invention is directed towards nucleic acid molecule segments comprising a structural nucleic acid sequence. The structural nucleic acid sequence preferably is at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to SEQ ID NO:1. Alternatively, the structural nucleic acid sequence can be a nucleic acid sequence that hybridizes under stringent hybridization conditions to the reverse complement of SEQ ID NO:1. The structural nucleic acid sequence can be SEQ ID NO:1.

The invention is further directed towards an isolated nucleic acid molecule segment comprising a structural nucleic acid sequence which encodes an amino acid sequence. The amino acid sequence can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to SEQ ID NO:2. Alternatively, the amino acid sequence can be an amino acid sequence immunoreactive with an antibody prepared using SEQ ID NO:2 as an antigen, the antibody being immunoreactive with SEQ ID NO:2. The structural nucleic acid sequence can encode SEQ ID NO:2.

A further embodiment of the invention is directed towards recombinant vectors. The vector preferably comprises operatively linked in the 5′ to 3′ orientation: a promoter that directs transcription of a structural nucleic acid sequence, a structural nucleic acid sequence, and a 3′ transcription terminator. The structural nucleic acid sequence is preferably selected from the group consisting of a nucleic acid sequence having a percent identity to SEQ ID NO:1, a nucleic acid sequence that hybridizes under stringent hybridization conditions to the reverse complement of SEQ ID NO:1, a nucleic acid sequence which encodes an amino acid sequence having a percent identity to SEQ ID NO:2, and a nucleic acid sequence which encodes an amino acid sequence immunoreactive with an antibody prepared using SEQ ID NO:2 as an antigen, the antibody being immunoreactive with SEQ ID NO:2. The percent identity to either SEQ ID NO:1 or SEQ ID NO:2 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:1, or can encode SEQ ID NO:2.

A further embodiment of the invention is directed towards a recombinant host cell. The recombinant host cell comprises a structural nucleic acid sequence. The structural nucleic acid sequence is preferably selected from the group consisting of a nucleic acid sequence having a percent identity to SEQ ID NO:1, a nucleic acid sequence that hybridizes under stringent hybridization conditions to the reverse complement of SEQ ID NO:1, a nucleic acid sequence which encodes an amino acid sequence having a percent identity to SEQ ID NO:2, and a nucleic acid sequence which encodes an amino acid sequence immunoreactive with an antibody prepared using SEQ ID NO:2 as an antigen, the antibody being immunoreactive with SEQ ID NO:2. The percent identity to either SEQ ID NO:1 or SEQ ID NO:2 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:1, or can encode SEQ ID NO:2.

An additional embodiment of the invention is directed towards a recombinant host cell. The recombinant host cell preferably comprises a structural nucleic acid sequence. The structural nucleic acid sequence is preferably selected from the group consisting of a nucleic acid sequence having a percent identity to SEQ ID NO:1, a nucleic acid sequence that hybridizes under stringent hybridization conditions to the reverse complement of SEQ ID NO:1, a nucleic acid sequence which encodes an amino acid sequence having a percent identity to SEQ ID NO:2, and a nucleic acid sequence which encodes an amino acid sequence immunoreactive with an antibody prepared using SEQ ID NO:2 as an antigen, the antibody being immunoreactive with SEQ ID NO:2. The percent identity to either SEQ ID NO:1 or SEQ ID NO:2 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:1, or can encode SEQ ID NO:2. The copy number of the structural nucleic acid sequence in the recombinant host cell is preferably higher than the copy number of the structural nucleic acid sequence in a wild type host cell of the same species. The copy number of the structural nucleic acid sequence in the wild type host cell can be zero. The copy number of the structural nucleic acid sequence in the recombinant host cell can be any positive integer, such as 1, 2, 3, 4, and so on. The recombinant host cell can generally be any type of cell. The recombinant host cell can be a bacterial cell, fungal cell, insect cell, or mammalian cell. The bacterial cell can be an Escherichia coli cell. The fungal cell can be a Saccharomyces cerevisiae, Schizosaccharomyces pombe, or Pichia pastoris cell. The insect cell can be a baculovirus compatible insect cell, or a Spodoptera cell. The mammalian cell can be a cancer cell or CHO cell.

The invention is further directed towards an isolated protein comprising an amino acid sequence. The amino acid sequence can be selected from the group consisting of an amino acid having a percent identity to SEQ ID NO:2, and an amino acid sequence immunoreactive with an antibody prepared using SEQ ID NO:2 as an antigen, the antibody being immunoreactive with SEQ ID NO:2. The percent identity to SEQ ID NO:2 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The amino acid sequence can be SEQ ID NO:2.

Additionally, the invention is directed towards an antibody prepared using SEQ ID NO:2 as an antigen, wherein the antibody is immunoreactive with SEQ ID NO:2. The antibody can be a polyclonal antibody or a monoclonal antibody.

An additional embodiment of the invention is directed towards a method of preparing a recombinant host cell. The method preferably comprises selecting a host cell, transforming the host cell with a recombinant vector, and obtaining recombinant host cells. The recombinant vector preferably comprises a structural nucleic acid sequence selected from the group consisting of: a nucleic acid having a percent identity to SEQ ID NO:1, a nucleic acid sequence that hybridizes under stringent hybridization conditions to the reverse complement of SEQ ID NO:1, a nucleic acid sequence which encodes an amino acid sequence having a percent identity to SEQ ID NO:2, and a nucleic acid sequence which encodes an amino acid sequence immunoreactive with an antibody prepared using SEQ ID NO:2 as an antigen, the antibody being immunoreactive with SEQ ID NO:2. The percent identity to SEQ ID NO:1 or SEQ ID NO:2 can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:1, or can encode SEQ ID NO:2. The host cell can generally be any type of cell. The host cell can be a bacterial cell, fungal cell, insect cell, or mammalian cell. The bacterial cell can be an Escherichia coli cell. The fungal cell can be a Saccharomyces cerevisiae, Schizosaccharomyces pombe, or Pichia pastoris cell. The insect cell can be a baculovirus compatible insect cell, or a Spodoptera cell. The mammalian cell can be a cancer cell or CHO cell.

CMG-2 Related Inventions

One embodiment of the invention is directed towards nucleic acid molecule segments comprising a structural nucleic acid sequence. The structural nucleic acid sequence preferably is at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to SEQ ID NO:3. Alternatively, the structural nucleic acid sequence can be a nucleic acid sequence that hybridizes under stringent hybridization conditions to the reverse complement of SEQ ID NO:3. The structural nucleic acid sequence can be SEQ ID NO:3.

The invention is further directed towards an isolated nucleic acid molecule segment comprising a structural nucleic acid sequence which encodes an amino acid sequence. The amino acid sequence can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to SEQ ID NO:4. Alternatively, the amino acid sequence can be an amino acid sequence immunoreactive with an antibody prepared using SEQ ID NO:4 as an antigen, the antibody being immunoreactive with SEQ ID NO:4. The structural nucleic acid sequence can encode SEQ ID NO:4.

A further embodiment of the invention is directed towards recombinant vectors. The vector preferably comprises operatively linked in the 5′ to 3′ orientation: a promoter that directs transcription of a structural nucleic acid sequence, a structural nucleic acid sequence, and a 3′ transcription terminator. The structural nucleic acid sequence is preferably selected from the group consisting of a nucleic acid sequence having a percent identity to SEQ ID NO:3, a nucleic acid sequence that hybridizes under stringent hybridization conditions to the reverse complement of SEQ ID NO:3, a nucleic acid sequence which encodes an amino acid sequence having a percent identity to SEQ ID NO:4, and a nucleic acid sequence which encodes an amino acid sequence immunoreactive with an antibody prepared using SEQ ID NO:4 as an antigen, the antibody being immunoreactive with SEQ ID NO:4. The percent identity to either SEQ ID NO:3 or SEQ ID NO:4 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:3, or can encode SEQ ID NO:4.

A further embodiment of the invention is directed towards a recombinant host cell. The recombinant host cell comprises a structural nucleic acid sequence. The structural nucleic acid sequence is preferably selected from the group consisting of a nucleic acid sequence having a percent identity to SEQ ID NO:3, a nucleic acid sequence that hybridizes under stringent hybridization conditions to the reverse complement of SEQ ID NO:3, a nucleic acid sequence which encodes an amino acid sequence having a percent identity to SEQ ID NO:4, and a nucleic acid sequence which encodes an amino acid sequence immunoreactive with an antibody prepared using SEQ ID NO:4 as an antigen, the antibody being immunoreactive with SEQ ID NO:4. The percent identity to either SEQ ID NO:3 or SEQ ID NO:4 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:3, or can encode SEQ ID NO:4.

An additional embodiment of the invention is directed towards a recombinant host cell. The recombinant host cell preferably comprises a structural nucleic acid sequence. The structural nucleic acid sequence is preferably selected from the group consisting of a nucleic acid sequence having a percent identity to SEQ ID NO:3, a nucleic acid sequence that hybridizes under stringent hybridization conditions to the reverse complement of SEQ ID NO:3, a nucleic acid sequence which encodes an amino acid sequence having a percent identity to SEQ ID NO:4, and a nucleic acid sequence which encodes an amino acid sequence immunoreactive with an antibody prepared using SEQ ID NO:4 as an antigen, the antibody being immunoreactive with SEQ ID NO:4. The percent identity to either SEQ ID NO:3 or SEQ ID NO:4 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:3, or can encode SEQ ID NO:4. The copy number of the structural nucleic acid sequence in the recombinant host cell is preferably higher than the copy number of the structural nucleic acid sequence in a wild type host cell of the same species. The copy number of the structural nucleic acid sequence in the wild type host cell can be zero. The copy number of the structural nucleic acid sequence in the recombinant host cell can be any positive integer, such as 1, 2, 3, 4, and so on. The recombinant host cell can generally be any type of cell. The recombinant host cell can be a bacterial cell, fungal cell, insect cell, or mammalian cell. The bacterial cell can be an Escherichia coli cell. The fungal cell can be a Saccharomyces cerevisiae, Schizosaccharomyces pombe, or Pichia pastoris cell. The insect cell can be a baculovirus compatible insect cell, or a Spodoptera cell. The mammalian cell can be a cancer cell or CHO cell.

The invention is further directed towards an isolated protein comprising an amino acid sequence. The amino acid sequence can be selected from the group consisting of an amino acid having a percent identity to SEQ ID NO:4, and an amino acid sequence immunoreactive with an antibody prepared using SEQ ID NO:4 as an antigen, the antibody being immunoreactive with SEQ ID NO:4. The percent identity to SEQ ID NO:4 mentioned above can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The amino acid sequence can be SEQ ID NO:4.

Additionally, the invention is directed towards an antibody prepared using SEQ ID NO:4 as an antigen, wherein the antibody is immunoreactive with SEQ ID NO:4. The antibody can be a polyclonal antibody or a monoclonal antibody.

An additional embodiment of the invention is directed towards a method of preparing a recombinant host cell. The method preferably comprises selecting a host cell, transforming the host cell with a recombinant vector, and obtaining recombinant host cells. The recombinant vector preferably comprises a structural nucleic acid sequence selected from the group consisting of: a nucleic acid having a percent identity to SEQ ID NO:3, a nucleic acid sequence that hybridizes under stringent hybridization conditions to the reverse complement of SEQ ID NO:3, a nucleic acid sequence which encodes an amino acid sequence having a percent identity to SEQ ID NO:4, and a nucleic acid sequence which encodes an amino acid sequence immunoreactive with an antibody prepared using SEQ ID NO:4 as an antigen, the antibody being immunoreactive with SEQ ID NO:4. The percent identity to SEQ ID NO:3 or SEQ ID NO:4 can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The structural nucleic acid sequence can be SEQ ID NO:3, or can encode SEQ ID NO:4. The host cell can generally be any type of cell. The host cell can be a bacterial cell, fungal cell, insect cell, or mammalian cell. The bacterial cell can be an Escherichia coli cell. The fungal cell can be a Saccharomyces cerevisiae, Schizosaccharomyces pombe, or Pichia pastoris cell. The insect cell can be a baculovirus compatible insect cell, or a Spodoptera cell. The mammalian cell can be a cancer cell or CHO cell.

An alternative embodiment of the invention relates to an isolated fusion protein. The fusion protein preferably comprises a first amino acid sequence having a percent identity to SEQ ID NO:5, and a second amino acid sequence. The percent identity can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The first amino acid sequence can be SEQ ID NO:4 or SEQ ID NO:5. The second amino acid sequence can be a polyhistidine tag sequence or a green fluorescent protein (GFP) sequence.

The invention further relates to a method of isolating a matrix protein. The method preferably comprises contacting the matrix protein and a CMG protein to produce a matrix protein—CMG protein complex, isolating the matrix protein—CMG protein complex, dissociating the matrix protein—CMG protein complex, and obtaining an isolated the matrix protein. The CMG protein preferably comprises an amino acid sequence selected from the group consisting of an amino acid sequence having a percent identity to SEQ ID NO:4, an amino acid sequence having a percent identity to SEQ ID NO:5, an amino acid sequence immunoreactive with an antibody prepared using SEQ ID NO:4 as an antigen, the antibody being immunoreactive with SEQ ID NO:4, and an amino acid sequence immunoreactive with an antibody prepared using SEQ ID NO:5 as an antigen, the antibody being immunoreactive with SEQ ID NO:5. The percent identity to SEQ ID NO:4 or to SEQ ID NO:5 can be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identity. The amino acid sequence can be SEQ ID NO:4 or SEQ ID NO:5. The matrix protein can be collagen type IV or laminin.

Regulation of Angiogenesis

Angiogenesis can be positively or negatively regulated using the nucleic acid and amino acid sequences disclosed herein. A method of regulating angiogenesis can comprise contacting cells with an viral vector encoding a CMG-2 protein or a CMG-2 protein fragment. Positive or negative regulation can be determined by measuring the rate of angiogenesis of the contacted cells as compared to the measured rate of angiogenesis of uncontacted cells. The regulation can be expressed as a ratio or percentage. The CMG-2 protein fragment can be a truncation from the N-terminus, C-terminus, or from both the N-terminus and C-terminus. The cells can be mammalian cells, preferably are human cells, dog cells, cat cells, pig cells, monkey cells, cow cells, horse cells, sheep cells, bear cells, or moose cells, and more preferably are human cells. The contacting step can be performed in vitro or in vivo, and preferably is performed in vivo. The contacting step can be achieved by any type of delivery, and preferably is by IV, IP, IM, transdermal, intranasal, or oral delivery. The CMG-2 protein is preferably SEQ ID NO:4. The viral vector preferably comprises SEQ ID NO:3.

An alternative method of regulating angiogenesis can comprise contacting cells with an antisense nucleic acid molecule, wherein the antisense nucleic acid molecule hybridizes to mRNA encoding a CMG-2 protein. The mRNA can be transcribed from a nucleic acid sequence in the cells comprising SEQ ID NO:3. The CMG-2 protein is preferably SEQ ID NO:4. The method can further comprise contacting the cells with a viral vector encoding the antisense nucleic acid molecule. The viral vector can generally be any kind of viral vector, and preferably is an adenoviral vector.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Materials

The GFP-N2 vector was from Clontech, while the pAdEasy adenoviral system was kindly provided by Dr. Bert Vogelstein and Dr. Tong-Chuan He (Johns Hopkins School of Medicine, Baltimore, Md.). A monoclonal antibody directed to hsp47 (Cates et al., 1987) was the kind gift of Dr. B. D. Sanwal (University of Western Ontario, London, Canada). Oligonucleotide primers were synthesized by Sigma-Genosys (The Woodlands, Tex.). The pQE30 vector and Ni/Cd-sepharose were from Qiagen (Valencia, Calif.). Antibodies to laminin were obtained from Sigma (St. Louis, Mo.), anti-Idl was from Upstate Biotechnologies (Lake Placid, N.Y.), anti-collagen type IV from Chemicon (Temecula, Calif.), and anti-α2 macroglobulin from ICN (Costa Mesa, Calif.). Peroxidase-conjugated rabbit anti-mouse IgG and goat anti-rabbit IgG antibodies were from Dako (Carpinteria, Calif.) and chemiluminescence reagents were from Amersham (Piscataway, N.J.). Rhodamine-conjugated rabbit anti-mouse IgG and fluorescein-conjugated goat anti-rabbit IgG were from Dako.

### Example 2

### Capillary Morphogenesis Assay

Human umbilical vein endothelial cells (Clonetics, San Diego, Calif.) were cultured as described (Maciag et al., 1979). EC cultures in three-dimensional collagen matrices were performed as described (Davis and Camarillo, 1996; Salazar et al., 1999) except that ECs (passages 2-5) were seeded at 2×10⁶ cells per ml of gel.

### Example 3

### DNA Microarray Analysis

DNA microarray analysis (DeRisi et al., 1997; Iyer et al., 1999) was used to study genomic-scale gene expression comparing four time points during capillary morphogenesis. Total RNA was extracted (Chomczynski and Sacchi, 1987) from ECs in the collagen gel, after collagenase treatment, using TRIzol reagent (Life Technologies, Grand Island, N.Y.) at 0, 8, 24, and 48 hour time points. Approximately 360 gels for each time point were needed to obtain enough mRNA for this experiment. Total RNA was passed twice through Oligotex beads to obtain mRNA (Qiagen). The poly-A RNA was eluted in DEPC-H₂O and sent to Incyte Genomics (St. Louis, Mo.) who performed the differential hybridization to a Unigem V chip containing 7,075 genes comparing 0 hr to 8 hr, 0 hr to 24 hr and 0 hr to 48 hr RNA samples. The data presented are ratios of hybridization between these time points.

### Example 4

### Reverse Transcription Polymerase Chain Reaction (RTPCR)

Total RNA was used to create cDNA templates and was equalized between the time points by spectrophotometry and formaldehyde agarose gel electrophoresis. Total RNA (5 μg) was used for reverse transcription (Stratagene, La Jolla, Calif.) to create random-primed cDNA at 0, 8, 24, and 48 hours of culture progression. RT-PCR amplification parameters used were typically 94° C. for 45 seconds, 60° C. for 45 seconds, 72° C. for 2 minutes; this was cycled 25 to 35 times, depending upon the gene, with a final extension at 72° C. for 5 minutes using an PTC-100 thermal cycler (MJ Research, Watertown, Mass.).

### Example 5

### Northern and Western Blot Analyses

Northern blot analyses were performed using total RNA (see above) equalized by spectrophotometry (3 μg per lane) from 0, 8, 24, and 48 hour time points as described (Salazar et al., 1999). Collagen gels were removed from wells, placed directly into boiling SDS sample buffer and heated to 100° C. for 10 minutes, and stored at −20° C. until use. Cell extracts were run on standard SDS-PAGE gels or 7% Blattler SDS-PAGE gels (collagen IV, laminin) (Blattler et al., 1972), and blots were incubated and developed as described previously (Salazar et al., 1999).

### Example 6

### Differential Display Analysis

Differential display protocols (GenHunter, Nashville, Tenn.) were used to identify genes that are differentially regulated during capillary morphogenesis (Liang and Pardee, 1998; Martin and Pardee, 1999). A cDNA copy of the total RNA (obtained as above) was created using random primers from 0, 8, 24, and 48 hour time points. This cDNA amplification was performed using combinations of three downstream oligo dT primers, G-T₁₁M, C-T₁₁M, and A-T₁₁M, and a series of random 10 mer upstream primers, AP-1 through AP-80 (ten sets of eight). We used sets one and two of the random upstream primers (AP-1 through AP-16) in combination with oligo dT downstream primers (GenHunter) which represent about 20% of the total primer combinations. Forty cycles of amplification, incorporating [γ-³³P]-dCTP, was used to create the random fragments. The fragments were then run on a 6% polyacrylamide sequencing gel using 1× TAE as the running buffer, and resolved by autoradiography. Differentially expressed bands were excised, boiled to extract DNA, and ethanol precipitated using glycogen as a carrier. Individual fragments were then amplified using the appropriate differential display primers appropriate for that band and purified for further use. DNA fragments were TA cloned into pGEM-T-Easy (Promega, Madison, Wis.) and were sequenced by automated sequencing (Lonestar Laboratories, Houston, Tex.).

### Example 7

### Endothelial Cell Morphogenic cDNA Libraries

cDNA libraries were made using mRNA isolated from 8 and 24 hour cultures. Library production required 1 mg of total RNA (obtained as above) isolated from 13 ml of collagen gel containing 2×10⁶ cells/ml, which was aliquoted into 25 μl aliquots in 96 well A/2 microplates. After poly-A selection, first- and second-strand cDNA synthesis was performed with oligo dT primers. The cDNA was fractionated by size, and mass cloned into the ZAP-XR vector using the Uni-Zap XR Stratagene system. The cloning was performed unidirectionally, based on opposing EcoRi and XhoI restriction sites at the 5′ and 3′ ends, respectively. Each fraction was then packaged, and the first fraction was used for amplification while the remaining fractions were left unamplified using standard methodologies from Stratagene. These libraries were screened using ³²p labelled partial cDNAs to obtain larger clones (Wahl and Berger, 1987).

### Example 8

### Construction of Recombinant Adenoviruses

Recombinant adenoviral constructs were prepared essentially as described (He et al., 1998). Full length CMG-1 and CMG-2 were cloned into the GFP-N2 vector (Clontech) and were then amplified as GFP fusion protein constructs and then further cloned into the pAdShuttle-CMV vector. CMG-1 and CMG-2 were amplified and cloned into pEGFP-N2 (Clontech) using XhoI and BamH1 and XhoI and Eco R1 restriction sites, respectively. The following primer sets were used to amplify CMG-1 or CMG-2 inserts: 5′-AGCTCGAGACAATGGCCAGCAATCAC-3′ (SEQ ID NO:6) and 5′-AGGGATCCGGTTTCCGCTGGTGCTATG-3′ (SEQ ID NO:7) for CMG-1; 5′-AGCTCGAGAGGATGGTGGCGGAGCGGT-3′ (SEQ ID NO:8) and 5′-AGGAATTCAGCAGTTAGCTCTTTCTC-3′ (SEQ ID NO:9) for CMG-2.

For cloning of these cDNAs into the pShuttle-CMV vector, BglII and XbaI and XhoI and XbaI were used for CMG-1 and CMG-2, respectively. The common downstream primer 5′-AGTCTAGATTATGATCTAGAGTCGCGGC-3′ (SEQ ID NO:10) was used with the upstream primer 5′-AGAGATCTACAATGGCCAGCAATCAC-3′ (SEQ ID NO:11) for CMG-1 and 5′-AGCTCGAGAGGATGGTGGCGGAGCGGT-3′ (SEQ ID NO:12) for CMG-2.

These pShuttle-CMV clones were then recombined with pAdEasy-1 and were transfected into 293 cells to produce recombinant viruses. The viruses were then amplified through 3 passages in 293 cells before use. Extracts of 293 cells infected with these viruses were tested on SDS-PAGE gels and Western blots with anti-GFP antibodies showing that the fusion proteins were of appropriate molecular weight indicative of intact CMG-1-GFP and CMG-2-GFP fusion proteins. Endothelial cell monolayers were infected on gelatin coated coverslips for 4-5 hours in serum-free media and then this media was replaced with complete growth media overnight. After 24 hours, cultures were fixed with 3% paraformaldehyde and were either directly examined by fluorescence microscopy or were processed further for immunofluorescence staining as described (Salazar et al., 1999).

### Example 9

### Recombinant CMG-2 Production and Extracellular Matrix Protein Binding Assays

A portion of recombinant CMG-2 (residues 34-214) was produced in E. coli as a recombinant His-tagged protein. A CMG-2 cDNA was unidirectionally cloned into pQE30 through BamH1 and Hind III sites. Primers used to amplify CMG-2 were: 5′-AGGGATCCCAGGAGCAGCCCTCCTGC-3′ (SEQ ID NO:13) and 5′-AGAAGCTTAGAAGAATTAATTATTCC-3′ (SEQ ID NO:14). The recombinant protein was purified using Ni/Cd-sepharose as described (Bayless and Davis, 2001) and approximately 3 mg of protein was obtained from 400 ml of IPTG-induced. bacteria. Control GFP was also produced as a His-tagged protein and purified in the same way. Both proteins were adsorbed to plastic microwells at 10 μg/ml and after detergent blocking (0.1% Tween-Tris-saline, pH 7.5) for 30 minutes, biotinylated extracellular matrix proteins were added (1 μg/ml) in 0.1% Tween-Tris-saline containing 1% BSA for 1 hour. The biotinylated matrix proteins were prepared as described (Davis and Camarillo, 1993). After washing, the wells were further incubated with avidin-peroxidase at 1 μg/ml for 30 minutes in Tween-Tris-saline-BSA and after washing, were developed for peroxidase activity and read at 490 nm.

### Example 10

### Isolation and Identification of Novel Capillary Morphogenesis Genes (CMGs) by Differential Display and cDNA Library Screening

Novel sequences, whose messages were differentially regulated during EC morphogenesis, were isolated using differential display and cDNA library screening. We have termed the novel genes identified by these techniques, capillary morphogenesis genes (CMGs), which are defined as novel genes that are differentially expressed during the process of EC morphogenesis. A partial list of the genes identified by this analysis are shown in Table 1 with expression patterns.

TABLE 2
Expression patterns

| Name | 0 h | 8 h | 24 h | 48 h |
|---|---|---|---|---|
| CMG-1 | +++ | + | ++ | +++ |
| CMG-2 | + | +++ | ++ | + |
| CMG-3 | + | + | ++ | + |
| CMG-4 | +++ | ++ | + | − |
| CMG-5 | +/− | +++ | + | +/− |
| CMG-6 | +/− | +++ | + | +/− |

A number of known differentially regulated genes were identified in this analysis including melanoma-associated antigen (an extracellular matrix-like protein with RGD site/peroxidase like domains), tissue factor pathway inhibitor-2 (an extracellular matrix-associated serine proteinase inhibitor), germinal center protein kinase related kinase-1 (in gene family of proteins with functions in MAP kinase, Rho GTPase family signalling), melanin concentrating hormone (regulates ion/water transport across membranes), prothymosin-α (nuclear protein, implicated in cell proliferation), NADH-ubiquinone oxidoreductase-B 12 subunit (enzyme in the electron-transport chain), sodium bicarbonate cotransporter-3 (Pushkin et al., 1999; regulates intracellular/extracellular pH), NIP-2 (Brusadelli et al., 2000; bcl2-interacting protein) and Fte-1 (v-fos transformation effector gene). Melanoma-associated antigen (MG50) (Mitchell et al., 2000) is markedly upregulated (pattern C) during morphogenesis while the plasmin and serine proteinase inhibitor, TFPI-2, is strongly upregulated early in the time course (pattern B). Interestingly, melanin concentrating hormone and another member of the sodium bicarbonate cotransporter family (cotransporter-2 versus cotransporter-3) were identified as being differentially regulated as well as by DNA microarray analysis.

To confirm the expression patterns for genes isolated by differential display analysis, RT-PCR, Northern blot and Western blot analyses were performed. These results indicated that the CMGs and other genes are differentially expressed during EC morphogenesis.

### Example 11

### A Differentially Expressed Capillary Morphogenesis Gene, CMG-1, Contains Coiled-coil Domains and Targets to an Intracellular Vesicular Compartment

The full length sequence of CMG-1 encodes a putative intracellular 65 kDa protein. The sequence reveals a series of coiled-coil domains (from residues 96 to 560) which in other proteins have been reported to participate in protein-protein binding, protein multimerization, vesicular fusion and other functions (Burkhard et al., 2001). CMG-1 also contains several consensus motifs for phosphorylation including two for tyrosine phosphorylation at residues 96/97 and 572, one for cAMP/cGMP protein kinases at residue 260, and multiple protein kinase C and casein kinase II sites. Homology searches revealed the greatest similarity (24% identity, 46% positives from residues 9-591) with a putative C. elegans protein, C18H9.8. It also shows 24% identity from residues 104-592 (in the coiled coil domain) with myosin heavy chain sequences from various species. The sequence also matches human genome sequences and maps to human chromosome 9q. Interestingly, the pattern of CMG-1 gene expression during EC morphogenesis mirrors that of caveolin, and other major EC genes. Both genes show a marked downregulation at 8 hours of morphogenesis followed by a return to baseline by 48 hours. To examine the expression pattern of CMG-1 in adult versus fetal human tissues, RT-PCR was performed. The strongest expression was observed from adult and fetal kidney cDNAs with detectable expression in adult heart, placenta, lung, liver and pancreas. Minimal to no expression was observed in the adult brain or skeletal muscle cDNA samples. Detectable expression was observed in fetal skeletal muscle as well as fetal heart, lung and liver, while minimal to no expression was observed from fetal brain, thymus and spleen cDNAs.

To reveal possible functions for CMG-1, a CMG-1-green fluorescent protein (GFP) protein chimera was constructed to assess where the protein targets intracellularly. Transfection of 293 epithelial tumor cells revealed targeting of the CMG-1 fusion protein to an intracellular vesicular compartment. To accomplish this experiment in human ECs, a recombinant adenovirus was constructed in the pAdEasy system carrying the CMG-1-GFP fusion protein. Infection of ECs resulted in an apparent intracellular distribution identical to that observed in 293 cells with targeting to multiple intracellular vesicles. In contrast, control GFP distributes throughout 293 cells or ECs with a cytoplasmic staining pattern. Coimmunostaining of ECs expressing CMG-1-GFP using antibodies to various known intracellular compartments such as endosomes, Weibel-Palade bodies, caveolae, mitochondria, Golgi apparatus (GM130), and lysosomes failed to reveal any colocalization.

### Example 12

### A Differentially Expressed Capillary Morphogenesis Gene, CMG-2, Contains a Putative Transmembrane Domain, Targets to the Endoplasmic Reticulum and Shows Affinity for the Basement Membrane Matrix Proteins, Collagen Type IV and Laminin

CMG-2 is markedly upregulated at 8 hr during EC morphogenesis as revealed by both RT-PCR and Northern blots. This nucleic acid sequence encodes a putative 45 kDa protein with a putative transmembrane segment and a potential signal peptide (residues 1-33). Polyclonal antibodies directed to recombinant CMG-2 were prepared, affinity purified and probed on Western blots of ECs undergoing morphogenesis. Induced protein bands migrating at the predicted size of 45 kDa were detected using this antibody. This antibody also specifically detects CMG-2-GFP or CMG-2-myc epitope-tagged fusion proteins by immunoprecipitation or immunoblotting demonstrating specificity for CMG-2. In contrast, G3PDH or actin antibodies show stable expression during the time course.

The CMG-2 gene maps to the human genome sequence and is located on chromosome 4q. Using the PSORT II program, the protein was predicted to have a 44% probability of targeting to the endoplasmic reticulum membrane with lesser probabilities to the Golgi apparatus or plasma membrane. Proximal to the potential transmembrane segment, homology searches reveal a von Willebrand Factor A domain (a matrix-binding domain) from residues 44-213. In addition, WH-1 block homologies were detected to WASP, a cdc42-binding protein that regulates the actin cytoskeleton (Anton et al., 1998) (from residues 250-259 and 315-334). The human tissue distribution of CMG-2 was assessed by RT-PCR. CMG-2 was detected in placenta but was not detected in the other adult or fetal tissues examined.

To address where CMG-2 may target within ECs, the same approach described above was performed using a recombinant adenovirus carrying a CMG-2-GFP fusion protein. ECs were infected revealing that CMG-2-GFP primarily targets to endoplasmic reticulum (ER) using fluorescence microscopy. A double staining experiment using the ER protein, Hsp47, which is a chaperone protein for collagens type I and IV was performed (Clarke et al., 1991;Hendershot and Bulleid, 2000; Nagai et al., 2000). The staining pattern did not overlap with a Golgi-specific marker. Additionally, CMG-2-GFP was observed to be present within intracellular vesicles in some cells suggesting that it may be capable of cycling from the ER to intracellular vesicles or that it may separately target to more than one compartment. Targeting of CMG-2-GFP to the plasma membrane has not yet been observed.

A 20 kDa portion of the CMG-2 protein with sequence homology to the Von Willebrand factor A domain was expressed in bacteria and tested for its ability to bind extracellular matrix proteins. The recombinantly expressed protein along with a control GFP recombinant protein were purified using their histidine tags. These proteins were adsorbed to plastic and were incubated with biotinylated collagen type IV, laminin, fibronectin, osteopontin and control albumin. The CMG-2 protein but not the control GFP protein showed strong binding to the basement membrane proteins, collagen type IV and laminin, but showed little to no binding affinity for the other ECM proteins. This data suggests that CMG-2 has affinity for matrix proteins which implies a potential role in basement membrane matrix synthesis or assembly due to its localization within the endoplasmic reticulum of ECs.

### Example 13

### Novel Capillary Morphogenesis Genes (CMGs) are Differentially Expressed During EC Morphogenesis in Three-dimensional Collagen Matrices

One of the clear advantages of the inventors' published system (Davis and Camarillo, 1996) is its utility for the identification of differentially expressed known and novel genes in capillary morphogenesis (Salazar et al., 1999; Kahn et al., 2000; Davis et al., 2001). Other EC morphogenic models have also been used to study differential gene expression (Glienke et al., 2000). Here, a combination of experimental approaches have been used to screen large numbers of genes for differential expression patterns. In addition, the initial characterization of a number of genes that were isolated using differential display and cDNA library screening is presented.

Here, the full length sequences of CMG-1 and CMG-2 are presented, with coding sequences predicting proteins of 65 kDa and 45 kDa, respectively (S. E. Bell, et al. J. Cell Sci. 114: 2755-2773, 2001; GenBank Accession Nos. AY040325 and AY040326). CMG-1 is predicted to be intracellular and to contain a series of coiled-coil domains involving ˜500 amino acids of sequence. A CMG-1-GFP construct was observed to target to an intracellular vesicular compartment. Interestingly, it has an expression pattern which mirrors that of caveolin-1, endothelin-1, and ICAM-2. RT-PCR analysis of tissue expression reveals its mRNA expression in a number of tissues with the most abundant being adult and fetal human kidney. CMG-2 contains a putative transmembrane domain and signal peptide and was predicted to target to the endoplasmic reticulum which was confirmed using a CMG-2-GFP fusion protein vector. Its affinity for basement membrane ECM proteins suggests a potential role in basement membrane matrix synthesis and assembly in ECs during morphogenesis. CMG-2 mRNA was detected in placenta and was essentially undetectable in the other adult and fetal tissues examined. Thus, CMG-2 appears to have a much more restricted tissue distribution than CMG-1.

### Example 14

### Positive and Negative Regulation of Angiogenesis using CMG-2 Sequences

The CMG-2 nucleic acid sequence is differentially expressed during human blood vessel formation (commonly referred to as angiogenesis). Additionally, it is expressed by angiogenic blood vessels in human tissues that are undergoing tissue repair as detected by in situ hybridization. The CMG-2 protein has been shown to bind to collagen type IV and to laminin. Regulating the concentration or function of the CMG-2 protein within endothelial cells may lead to either the stimulation or inhibition of angiogenesis. This regulation would affect diseases involving tumor growth/spread, or diseases with chronic injury such as arthritis, atherosclerosis, and diabetes.

The CMG-2 protein, or a fragment thereof can be administered directly to a tumor or other site of interest. The administration can be by injection (IV, IP, IM, topical), or by a facilitated delivery such as vessicles or transdermal delivery. The concentration of the CMG-2 protein can be increased by use of a vector system such as by transformation with an adenoviral vector encoding the CMG-2 protein. The concentration of the CMG-2 protein can be decreased by the use of antisense technology, where a nucleic acid sequence designed to hybridize to the CMG-2 mRNA is delivered either systemically or locally to the region of the tumor or other sites of interest.

The use of a CMG-2 protein fragment, or the use of a nucleic acid encoding such a protein fragment can be used as an alternative to the full length protein or nucleic acid. The protein fragment may bind to a receptor, and compete with the native full length CMG-2 protein. The full length CMG-2 protein has several distinct domains including: an export peptide sequence (amino acids 1-33, SEQ ID NO:17), an Integrin a subunit I-domain (amino acids 34-207, SEQ ID NO:18), a vWF A-domain (amino acids 142-150 SEQ ID NO:19), a transmembrane domain (amino acids 215-231, SEQ ID NO:20), and a WASP WH-1 domain (amino acids 250-259, SEQ ID NO:21). A CMG-2 protein fragment can comprise one or more of these domains.

### Example 15

### Alternative Nucleic Acid and Protein Sequences

For future variations of the CMG-1 and CMG-2 proteins, coding sequences for CMG-1 and CMG-2 from other organisms could be used in producing CMG proteins. Other coding sequences to be used in this way could be identified by such methods as database similarity or homology searches, functional activity of the proteins being similar to CMG-1 and CMG-2 proteins, crystallographic studies of proteins similar in structure or function, by hybridization to probes designed to find such coding sequences, or synthetic coding sequences designed to produce the protein product of such coding sequences.

Sources other than human cells may be used to obtain the CMG-1 or CMG-2 nucleic acid sequence, and the encoded CMG-1 or CMG-2 protein. For example, sequences from other mammals such as dogs, cats, pigs, monkeys, cows, horses, sheep, bears, or moose can be used. Furthermore, subunit sequences from different organisms may be combined to create a novel CMG-1 or CMG-2 sequence incorporating structural, regulatory, and enzymatic properties from different sources.

### Example 16

### Nucleic Acid Mutation and Hybridization

Variations in the nucleic acid sequence encoding a CMG-1 or CMG-2 protein may lead to mutant CMG-1 and CMG-2 protein sequences that display equivalent or superior enzymatic characteristics when compared to the sequences disclosed herein. This invention accordingly encompasses nucleic acid sequences which are similar to the sequences disclosed herein, protein sequences which are similar to the sequences disclosed herein, and the nucleic acid sequences that encode them. Mutations may include deletions, insertions, truncations, substitutions, fusions, shuffling of subunit sequences, and the like.

Mutations to a nucleic acid sequence may be introduced in either a specific or random manner, both of which are well known to those of skill in the art of molecular biology. A myriad of site-directed mutagenesis techniques exist, typically using oligonucleotides to introduce mutations at specific locations in a nucleic acid sequence. Examples include single strand rescue (Kunkel, T. Proc. Natl. Acad. Sci. U.S.A., 82: 488-492, 1985), unique site elimination (Deng and Nickloff, Anal. Biochem. 200: 81, 1992), nick protection (Vandeyar, et al. Gene 65: 129-133, 1988), and PCR (Costa, et al. Methods Mol. Biol. 57: 31-44, 1996). Random or non-specific mutations may be generated by chemical agents (for a general review, see Singer and Kusmierek, Ann. Rev. Biochem. 52: 655-693, 1982) such as nitrosoguanidine (Cerda-Olmedo et al., J. Mol. Biol. 33: 705-719, 1968; Guerola, et al. Nature New Biol. 230: 122-125, 1971) and 2-aminopurine (Rogan and Bessman, J. Bacteriol. 103: 622-633, 1970), or by biological methods such as passage through mutator strains (Greener et al. Mol. Biotechnol. 7: 189-195, 1997).

Nucleic acid hybridization is a technique well known to those of skill in the art of DNA manipulation. The hybridization properties of a given pair of nucleic acids is an indication of their similarity or identity. Mutated nucleic acid sequences may be selected for their similarity to the disclosed CMG-2 nucleic acid sequences on the basis of their hybridization to the disclosed sequences. Low stringency conditions may be used to select sequences with multiple mutations. One may wish to employ conditions such as about 0.15 M to about 0.9 M sodium chloride, at temperatures ranging from about 20° C. to about 55° C. High stringency conditions may be used to select for nucleic acid sequences with higher degrees of identity to the disclosed sequences. Conditions employed may include about 0.02 M to about 0.15 M sodium chloride, about 0.5% to about 5% casein, about 0.02% SDS and/or about 0.1% N-laurylsarcosine, about 0.001 M to about 0.03 M sodium citrate, at temperatures between about 50° C. and about 70° C. More preferably, high stringency conditions are 0.02 M sodium chloride, 0.5% casein, 0.02% SDS, 0.001 M sodium citrate, at a temperature of 50° C.

### Example 17

### Determination of Homologous and Degenerate Nucleic Acid Sequences

Modification and changes may be made in the sequence of the proteins of the present invention and the nucleic acid segments which encode them and still obtain a functional molecule that encodes a protein with desirable properties. The following is a discussion based upon changing the amino acid sequence of a protein to create an equivalent, or possibly an improved, second-generation molecule. The amino acid changes may be achieved by changing the codons of the nucleic acid sequence, according to the codons given in Table 3.

TABLE 3
Codon degeneracies of amino acids

| | One | | |
|---|---|---|---|
| Amino acid | letter | Three letter | Codons |
| Alanine | A | Ala | GCA GCC GCG GCT |
| Cysteine | C | Cys | TGC TGT |
| Aspartic acid | D | Asp | GAC GAT |
| Glutamic acid | B | Glu | GAA GAG |
| Phenylalanine | F | Phe | TTC TTT |
| Glycine | G | Gly | GGA GGC GGG GGT |
| Histidine | H | His | CAC CAT |
| Isoleucine | I | lie | ATA ATC ATT |
| Lysine | K | Lys | AAA AAG |
| Leucine | L | Leu | TTA TTG CTA CTC CTG CTT |
| Methionine | M | Met | ATG |
| Asparagine | N | Asn | AAC AAT |
| Proline | P | Pro | CCA CCC CCG CCT |
| Glutamine | Q | Gin | CAA CAG |
| Arginine | R | Arg | AGA AGG CGA CGC CGG CGT |
| Serine | S | Ser | AGC AGT TCA TCC TCG TCT |
| Threonine | T | Thr | ACA ACC ACG ACT |
| Valine | V | Val | GTA GTC GTG GTT |
| Tryptophan | W | Trp | TGG |
| Tyrosine | Y | Tyr | TAC TAT |

Certain amino acids may be substituted for other amino acids in a protein sequence without appreciable loss of enzymatic activity. It is thus contemplated that various changes may be made in the peptide sequences of the disclosed protein sequences, or their corresponding nucleic acid sequences without appreciable loss of the biological activity.

In making such changes, the hydropathic index of amino acids may be considered. The importance of the hydropathic amino acid index in conferring interactive biological function on a protein is generally understood in the art (Kyte and Doolittle, J. Mol. Biol., 157: 105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics. These are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biologically functional protein. In making such changes, the substitution of amino acids whose hydropathic indices are within ±2 is preferred, those within ±1 are more preferred, and those within ±0.5 are most preferred.

It is also understood in the art that the substitution of like amino acids may be made effectively on the basis of hydrophilicity. U.S. Pat. No. 4,554,101 (Hopp, T. P., issued Nov. 19, 1985) states that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4).

It is understood that an amino acid may be substituted by another amino acid having a similar hydrophilicity score and still result in a protein with similar biological activity, i.e., still obtain a biologically functional protein. In making such changes, the substitution of amino acids whose hydropathic indices are within ±2 is preferred, those within ±1 are more preferred, and those within ±0.5 are most preferred.

As outlined above, amino acid substitutions are therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine, and isoleucine. Changes which are not expected to be advantageous may also be used if these resulted in functional CMG-1 and CMG-2 proteins.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

### References

The following references, to the extent that they provide exemplary procedural or other details supplementary to those set forth herein, are specifically incorporated herein by reference.

Anton, I. M., Lu, W., Mayer, B. J., Ramesh, N., and Geha, R. S. (1998) The Wiskott-Aldrich syndrome protein-interacting protein (WIP) binds to the adaptor protein Nck. J. Biol. Chem. 273: 20992-20995.

Bayless, K. J., Salazar, R., and Davis, G. E. (2000) RGD-dependent vacuolation and lumen formation observed during endothelial cell morphogenesis in three-dimensional fibrin matrices involves the αvβ3 and α5β1 integrins. Am. J. Pathol. 156:1673-1683.

Bayless, K. J. and Davis, G. E. (2001) Identification of dual α4β1 integrin binding sites within a 38 amino acid domain in the N-terminal thrombin fragment of human osteopontin. J. Biol. Chem., in press.

Blattler, D. P., Garner, F., Van Slyke, K., and Bradley, A. (1972) Quantitative electrophoresis in polyacrylamide gels of 2 to 40%. J. Chromat. 64: 147-155.

Borth, W. (1992). α₂-Macroglobulin, a multifunctional binding protein with targeting characteristics. FASEB J. 6: 3345-3353.

Bounpheng, M. A., Dimas, J. J., Dodds, S. G. and Christy, B. A. (1999) Degradation of the Id proteins by the ubiquitin-proteasome pathway. FASEB J. 13: 2257-2264.

Bowman, E. P., Campbell, J. J., Druey, K. M., Scheschonka, A., Kehrl, J. H., and Butcher, E. C. (1998) Regulation of chemotactic and proadhesive responses to chemoattractant receptors by RGS (regulator of G-protein signaling) family members. J. Biol. Chem. 273: 28040-28048.

Bradham, D. M., Igarashi, A., Potter, R. L., and Grotendorst, G. R. (1991). Connective tissue growth factor: a cysteine-rich mitogen secreted by human vascular endothelial cells is related to the src-induced immediate early gene product CEF-10. J. Cell Biol. 114: 1285-1294.

Brown, P. O., and Botstein, D. (1999) Exploring the new world of the genome with DNA microarrays. Nat. Genet. 21: 33-37.

Brusadelli, A., Sialino, H., Piepoli, T., Pollio, G., and Maggi, A. (2000) Expression of the estrogen-regulated gene Nip2 during rat brain maturation. Int. J. Dev. Neurosci. 18: 317-320.

Burkhard, P., Strelkov, S. V., and Stetefeld, J. (2001) Coiled coils: a highly versatile protein folding motif. Trends Cell Biol. 11: 82-88.

Carmeliet, P., and Jain, R. K. (2000) Angiogenesis in cancer and other diseases. Nature 407: 249-257.

Cates, G. A., Nandan, D., Brickenden, A. M., and Sanwal, B. D. (1987) Differentiation defective mutants of skeletal myoblasts altered in a gelatin-binding glycoprotein. Biochem. Cell Biol. 65: 767-775.

Chomczynski, P., and Sacchi, N. (1987) Single-step method of RNA isolation by acid guanidinium thiocyanate-phenol-chloroform extraction. Anal. Biochem. 162: 156-159.

Clarke, E. P., Cates, G. A., Ball, E. H., and Sanwal, B. D. (1991) A collagen-binding protein in the endoplasmic reticulum of myoblasts exhibits relationship with serine protease inhibitors. J. Biol. Chem. 266: 17230-17235.

Conway, E. M., Collen, D., and Carmeliet, P. (2001) Molecular mechanisms of blood vessel growth. Cardiovasc. Res. 49: 507-521.

Davis, G. E., Black, S. M., and Bayless, K. J. (2000) Capillary morphogenesis during endothelial cell invasion of three-dimensional collagen matrices. In Vitro Cell. Develop. Biol. Animal, 36:513-519.

Davis, G. E., and Camarillo, C. W. (1993) Regulation of integrin-mediated myeloid cell adhesion to fibronectin: Influence of disulfide reducing agents, divalent cations and phorbol ester. J. Immunol. 151: 7138-7150.

Davis, G. E., and Camarillo, C. W. (1995) Regulation of endothelial cell morphogenesis by integrins, mechanical forces, and matrix guidance pathways. Exp. Cell Res. 216: 113-123.

Davis, G. E., and Camarillo, C. W. (1996) An α2β1 integrin-dependent pinocytic mechanism involving intracellular vacuole formation and coalescence regulates capillary lumen and tube formation in three-dimensional collagen matrix. Exp. Cell Res. 224: 39-51.

Davis, G. E., Pintar Allen, K. A., Salazar, R., and Maxwell, S. A. (2001) Matrix metalloproteinase-1 and -9 activation by plasmin regulates a novel endothelial cell-mediated mechanism of collagen gel contraction and capillary tube regression in three-dimensional collagen matrices. J. Cell Sci. 114: 917-930.

DeRisi, J. L., Iyer, V. R., and Brown, P. O. (1997) Exploring the metabolic and genetic control of gene expression on a genomic scale. Science 278: 680-686.

DeVries, L., Zheng, B., Fischer, T., Elenko, E., and Farquhar, M. G. (2000) The regulator of G protein signaling family. Ann. Rev. Pharmacol. Toxicol. 40: 235-271.

Dietrich, A., Brazil, D., Jensen, O. N., Meister, M., Schrader, M., Moomaw, J. F., Mann, M., Illenberger, D., and Gierschik, P. (1996) Isoprenylation of the G protein gamma subunit is both necessary and sufficient for beta gamma dimer-mediated stimulation of phospholipase C. Biochemistry 35: 15174-15182.

Druey, K. M., Blumer, K. J., Kang, V. H. and Kehrl, J. H. (1996) Inhibition of G-protein-mediated MAP kinase activation by a new mammalian gene family. Nature 379: 742-746.

Dulin, N. O., Sorokin, A., Reed, E., Elliott, S., Kehrl, J. H., and Dunn, M. J. (1999) RGS3 inhibits G protein-mediated signaling via translocation to the membrane and binding to G 11. Mol. Cell. Biol. 19: 714-723.

Folkman, J. (1995) Angiogenesis in cancer, vascular, rheumatoid and other disease. Nat. Med. 1: 27-31.

Funamoto, M., Fujio, Y., Kunisada, K., Negoro, S., Tone, E., Osugi, T., Hirota, H., Izumi, M., Yoshizaki, K., Walsh, K., Kishimoto, T., Yamauchi-Takihara, K. (2000) Signal transducer and activator of transcription 3 is required for glycoprotein 130-mediated induction of vascular endothelial growth factor in cardiac myocytes. J. Biol. Chem. 275: 10561-10566.

Giltay, R., Timpl, R., and Kostka, G. (1999) Sequence, recombinant expression and tissue localization of two novel extracellular matrix proteins, fibulin-3 and fibulin-4. Matrix Biol. 18: 469-480.

Glienke, J., Schmitt, A. O., Pilarsky, C., Hinzmann, B., WeiB, B., Rosenthal, A. and Thierauch, K. H. (2000). Differential gene expression by endothelial cells in distinct angiogenic states. Eur. J. Biochem. 267: 2820-2830.

Goepfert, C., Imai, M., Brouard, S., Csizmadia, E., Kaczmarek, E., and Robson, S. C. (2000) CD39 modulates endothelial cell activation and apoptosis. Mol. Med. 6: 591-603.

Gonias, S. L., Carmichael, A., Mettenburg, J. M., Roadcap, D. W., Irvin, W. P., and Webb, D. J. (2000) Identical or overlapping sequences in the primary structure of human alpha (2)-macroglobulin are responsible for the binding of nerve growth factor-beta, platelet-derived growth factor-BB, and transforming growth factor-beta. J. Biol. Chem. 275: 5826-5831.

Grootjans, J. J., Zimmermann, P., Reekmans, G., Smets, A., Degeest, G., Durr, J., and David, G. (1997) Syntenin, a PDZ protein that binds syndecan cytoplasmic domains. Proc. Natl. Acad. Sci. USA 94: 13683-13688.

Grotendorst, G. R. (1997). Connective tissue growth factor: a mediator of TGF-beta action on fibroblasts. Cytokine Growth Factor Rev. 8, 171-179.

Hacohen, N., Kramer, S., Sutherland, D., Hiromi, Y., and Krasnow, M. A. (1998). Sprouty encodes a novel antagonist of FGF signaling that patterns apical branching of the Drosophilia airways. Cell 92: 253-263.

Hawes, B. E., Kil, E., Green, B., O'Neill, K., Fried, S., and Graziano, M. P. (2000) The melanin-concentrating hormone receptor couples to multiple G proteins to activate diverse intracellular signaling pathways. Endocrinology 141: 4525-4532.

Heald, I., and Weis, I. (2000) Spindles get the ran around. Trends Cell Biol. 10: 1-4.

Hendershot, L. M., and Bulleid, N. J. (2000) Protein-specific chaperones: The role of hsp47 begins to gel. Curr. Biol. 10: R912-R915.

Holash, J., Wiegand, S. J., and Yancopoulos, G. D. (1999) New model of tumor angiogenesis: a dynamic balance between vessel regression and growth mediated by angiopoietins and VEGF. Oncogene 18: 5356-5362.

Hooper, W. C., Phillips, D. J., and Evatt, B. L. (1997) Endothelial cell protein S synthesis is upregulated by the complex of IL-6 and soluble IL-6 receptor. Thromb. Haemost. 77: 1014-1019.

Howell, B. J., Hoffman, D. B., Fang, G., Murray, A. W., and Salmon, E. D. (2000) Visualization of Mad2 dynamics at kinetochores, along spindle fibers, and at spindle poles in living cells. J. Cell Biol. 150: 1233-1250.

Ilan, N.; Mahooti, S.; Madri, J. A. Distinct signal transduction pathways are utilized during the tube formation and survival phases of in vitro angiogenesis. J. Cell Sci. 111: 3621-3631; 1998.

Imada, K., and Leonard, W. J. (2000) The Jak-STAT pathway. Mol. Immunol. 37: 1-11.

Ingber, D., and Folkman, J. (1988) Inhibition of angiogenesis through modulation of collagen metabolism. Lab. Invest. 59: 44-51.

Iruela-Arispe, M. L., Hasselaar, P., and Sage, E. H. (1991) Differential expression of extracellular proteins is correlated with angiogenesis in vitro. Lab. Invest. 64: 174-186.

Iyer, V. R., Eisen, M. B., Ross, D. T., Schuler, G., Moore, T., Lee, J. C., Trent, J. M, Staudt, L. M., Hudson, J., Bogulski, M. S., Lashkari, D., Shalon, D., Botstein, D., and Brown, P. O. (1999) The transcriptional program in the response of human fibroblasts to serum. Science 283: 83-87.

Jensen, L. S. (1989). α2-Macroglobulins: Structure, Shape, and Mechanism of Proteinase Complex Formation. J. Biol. Chem. 264: 11539-11542.

Jiang, W., Jimenez, G., Wells, N. J., Hope, T. J., Wahl, G. M., Hunter, T., and Fukunaga, R. (1998) PRC 1: a human mitotic spindle-associated CDK substrate protein required for cytokinesis. Mol. Cell 2: 877-885.

Kahn, J., Mehraban, F., Ingle, G., Xin, X., Bryant, J. E., Vehar, G., Schoenfeld, J., Grimaldi, C. J., Peale, F., Draksharapu, A., Lewin, D. A., and Gerritsen, M. E. (2000) Gene expression profiling in an in vitro model of angiogenesis. Am. J. Pathol. 156: 1887-1900.

Kakuta, Y., Sueyoshi, T., Negishi, M., Pedersen, L. C. (1999). Crystal structure of the sulfotransferase domain of the human heparan sulfate N-deacetylase/N-sulfotransferase 1. J. Biol. Chem. 274: 10673-10676.

Koepp, D. M., Harper, J. W., and Elledge, S. J. (1999) How the cyclin became a cyclin: regulated proteolysis in the cell cycle. Cell 97: 431-434.

Kortesmaa, J., Yurchenco, P., and Tryggvason, K. (2000) Recombinant laminin-8 (alpha (4)beta(1)gamma(1)). Production, purification, and interactions with integrins. J. Biol. Chem. 275: 14853-14859.

Krebs, D. L., and Hilton, D. J. (2000) SOCS: physiological suppressors of cytokine signaling. J. Cell Sci. 113: 2813-2819.

Lee, E. G., Boone, D. L., Chai, S., Libby, S. L., Chien, M., Lodolce, J. P., and Ma, A. (2000) Failure to regulate TNF-induced NF-kappaB and cell death response in A20-deficient mice. Science 289: 2350-2354.

Lee, S. H., Schloss, D. J., Jarvis, L., Krasnow, M. A., and Swain, J. L. (2001) Inhibition of angiogenesis by a mouse sprouty protein. J. Biol. Chem. 276: 4128-4133.

Lefebvre, O., Sorokin, L., Kedinger, M., and Simon-Assmann, P. (1999) Developmental expression and cellular origin of the laminin alpha2, alpha4, and alpha5 chains in the intestine. Dev. Biol. 210:135-150.

Liang, P., and Pardee, A. B. (1998) Differential display. A general protocol. Mol. Biotechnol. 10: 261-267.

Lindsell, C. E., Shawber, C. J., Boulter, J., and Weinmaster, G. (1995). Jagged: a mammalian ligand that activates Notch 1. Cell 80: 909-917.

Lyden, D., Young, A. Z., Zagzag, D., Yan, W., Gerald, W., O'Rielly, R., Bader, B. L., Hynes, R. O., Zhuang, Y., Manova, K., Benzera, R. (1999). Id1 and Id3 are required for neurogenesis, angiogenesis and vascularization of tumor xenografts. Nature 401:670-677.

Mandriota, S. J., and Pepper, M. S. (1998). Regulation of angiopoietin-2 mRNA levels in bovine microvascular endothelial cells by cytokines and hypoxia. Circ. Res. 83: 852-859.

Maragoudakis, M. E., Sarmonika, M., and Panoutsacopoulou, M. (1988) Inhibition of basement membrane synthesis prevents angiogenesis. J. Pharmacol. Exp. Therapeut. 244: 729-733.

Martin, K. J. and Pardee, A. B. (1999) Principles of differential display. Methods Enzymol. 303: 234-258.

Maxwell, S. A., and Davis, G. E. (2000) Differential gene expression in p53-mediated apoptosis-resistant vs. apoptosis-sensitive tumor cell lines. Proc. Natl. Acad. Sci. USA 97: 13009-13014.

Mentlein, R. (1999) Dipeptidyl-peptidase IV (CD26)—role in the inactivation of regulatory peptides. Regul. Pept. 85: 9-24.

Metzger, R. J., and Krasnow, M. A. (1999) Genetic control of branching morphogenesis. Science 284: 1635-1639.

Miner, J. H., Cunningham, J., and Sanes, J. R. (1998) Roles for laminin in embryogenesis: exencephaly, syndactyly, and placentopathy in mice lacking the laminin alpha5 chain. J. Cell Biol. 143: 1713-1723.

Mitchell, M. S., Kan-Mitchell, J., Minev, B., Edman, C., and Deans, R. J. (2000) A novel melanoma gene (MG50) encoding the interleukin 1 receptor antagonist and six epitopes recognized by human cytolytic T lymphocytes. Canc. Res. 60: 6448-6456.

Montesano, R.; Pepper, M. S.; Vassalli, J. D.; Orci, L. Modulation of angiogenesis in vitro. EXS 61: 129-136; 1992.

Montesano, R. and Orci, L. Tumor-promoting phorbol esters induce angiogenesis in vitro. Cell 42: 469-477; 1985.

Morrissette, N. S., Gold, E. S., Guo, J., Hamerman, J. A., Ozinsky, A., Bedian, V., and Aderem, A. A. (1999) Isolation and characterization of monoclonal antibodies directed against novel components of macrophage phagosomes. J. Cell Sci. 112: 4705-4713.

Nagai, N., Hosokawa, M., Itohara, S., Adachi, E., Matsushita, T., Hosokawa, N., and Nagata, K. (2000) Embryonic lethality of molecular chaperone hsp47 knockout mice is associated with defects in collagen biosynthesis. J. Cell Biol. 150: 1499-1506.

Nandan, D., Clarke, E. P., Ball, E. H., and Sanwal, B. (1990) Ethyl 3,4 dihydroxybenzoate inhibits myoblast differentiation: evidence for an essential role of collagen. J. Cell Biol. 110: 1673-1679.

Nelson, R. E., Fessler, L. I., Takagi, Y., Blumberg, B., Keene, D. R., Olson, P. F., Parker, C. G., and Fessler, J. H. (1994) Peroxidasin: a novel enzyme-matrix protein of Drosophila development. EMBO J. 13: 3438-3447.

Nicosia, R. F. and Ottinetti, A. (1990) Growth of microvessels in serum-free matrix culture of rat aorta. A quantitative assay of angiogenesis in vitro. Lab. Invest. 63: 115-122.

Nicosia, R. F., and Villaschi, S. (1999) Autoregulation of angiogenesis by cells of the vessel wall. Int. Rev. Cytol. 185: 1-43.

Nigg, E. A. (2001) Mitotic kinases are regulators of cell division and its checkpoints. Nat. Rev. 2: 21-32.

Nilsson, I., and Hoffmann, I. (2000) Cell cycle regulation by the Cdc25 phosphatase family. Prog. Cell Cycle Res. 4: 107-114.

Nor, J. E., Christensen, J., Mooney, D. J., and Polverini, P. J. (1999) Vascular endothelial growth factor (VEGF)-mediated angiogenesis is associated with enhanced endothelial cell survival and induction of Bcl-2 expression. Am. J. Pathol. 154: 375-384.

Norton, J. D. (2000) Id helix-loop-helix proteins in cell growth, differentiation and tumorigenesis. J. Cell Sci. 113: 3897-3905.

Nurse, P., Masui, Y., and Hartwell, L. (1998) Understanding the cell cycle. Nat. Med. 4: 1103-1106.

Olivier, L. M. and Krisans, S. K. (2000) Peroxisomal protein targeting and identification of peroxisomal targeting signals in cholesterol biosynthetic enzymes. Biochim. Biophys. Acta 1529: 89-102.

Ollendorff, V., Noguchi, T., deLapeyriere, O., and Birnbaum, D. (1994) The GARP gene encodes a new member of the family of leucine-rich repeat-containing proteins. Cell Growth Differentiation. 5: 213-219.

Olsen, H. S., Cepeda, M. A., Zhang, Q., Rosen, C. A., Vozzolo, B. L., and Wagner, G. F. (1996). Human stanniocalcin: A possible hormonal regulator of mineral metabolism. Proc. Natl. Acad. Sci. USA 93: 1792-1796.

Page, A. M., and Hieter, P. (1999) The anaphase-promoting complex: new subunits and regulators. Ann. Rev. Biochem. 68: 583-609.

Park, J. E., Chen, H. H., Winer, J., Houck, K. A., and Ferrara, N. (1994) Placenta growth factor. Potentiation of vascular endothelial growth factor bioactivity, in vitro and in vivo, an high affinity binding to Flt-i but not to Flk/KDR. J. Biol. Chem. 269: 25646-25654.

Perbal, B., Martinerie, C., Sainson, R., Werner, M., He, B., Roizman, B. (1999). The C-terminal domain of the regulatory protein NOVH is sufficient to promote interaction with fibulin 1C: a clue for role of NOVH in cell-adhesion signaling. Proc. Natl. Acad. Sci. USA. 96: 869-874.

Perou, C. M., Sorlie, T., Eisen, M. B., van de Rijn, M., Jeffrey, S. S., Rees, C. A., Pollack, J. R., Ross, D. T., Johnsen, H., Akslen, L. A. Fluge, O., Pergamenschikov, A., Williams, C., Zhu, S. X., Lonning, P. E., Borresen-Dale, A. L., Brown, P. O., and Botstein, D. (2000) Molecular portraits of human breast tumours. Nature 406: 747-752.

Pushkin, A., Yip, K. P., Clark, I., Abuladze, N., Kwon, T. H., Tsuruoka, S., Schwartz, G. J., Nielsen, S., and Kurtz, I. (1999) NBC3 expression in rabbit collecting duct: colocalization with vacuolar H+-ATPase. Am. J. Physiol. 277: F974-F981.

Rao, C. N., Reddy, P., Liu, Y., O'Toole, E., Reeder, D., Foster, D. C., Kisiel, W., and Woodley, D. T. (1996) Extracellular matrix-associated serine protease inhibitors (Mr 33,000, 31,000, and 27,000) are single-gene products with differential glycosylation: cDNA cloning of the 33-kDa inhibitor reveals its identity to tissue factor pathway inhibitor-2. Arch. Biochem. Biophys. 335: 82-92.

Rattner, A., Hsieh, J-C., Smallwood, P. M., Gilbert, D. J., Copeland, N. G., Jenkins, N. A., and Nathans, J. (1997) A family of secreted proteins contains homology to the cysteine-rich ligand-binding domain of frizzled receptors. Proc. Natl. Acad. Sci. USA 94: 2859-2863.

Reich, A., Sapir, A., and Shilo, B. (1999) Sprouty is a general inhibitor of receptor tyrosine kinase signaling. Development 126: 4139-4147.

Rudner, A. D., and Murray, A. W. (2000) Phosphorylation by cdc28 activates the cdc20-dependent activity of the anaphase-promoting complex. J. Cell Biol. 149: 1377-1390.

Saijonmaa, O., Nyman, T., Kosonen, R., and Fyhrquist, F. (2000) Induction of angiotensin-converting enzyme by oncostatin M in human endothelial cells. Cytokine 12: 1253-1256.

Salazar, R., Bell, S. E., and Davis, G. E. (1999) Coordinate induction of the actin cytoskeletal regulatory proteins gelsolin, vasodilator-stimulated phosphoprotein, and profilin during capillary morphogenesis in vitro. Exp. Cell Res. 249: 22-32.

Sasaki, T., Majamaa, K., and Uitto, J. (1987) Reduction of collagen production in keloid fibroblast cultures by ethyl-3,4-dihydroxybenzoate. J. Biol. Chem. 262: 9397-9403.

Seki, N., Sugano, S., Suzuki, Y., Nakagawara, A., Ohira, M., Muramatsu, M., Saito, T., Hori, T. (1998). Isolation tissue expression, and chromosomal assignment human RGS5, a novel G-protein signaling regulator gene. J. Hum. Genet. 43: 202-205.

Sephel, G. C., Kennedy, R., and Kudravi, S. (1996) Expression of capillary basement membrane components during sequential phases of wound angiogenesis. Matrix. Biol. 15: 263-279.

Serrano, M., Hannon, G. J., and Beach, D. (1993). A new regulatory motif in cell-cycle control causing specific inhibition of cyclin D/CDK4. Nature. 366: 704-707.

Shirogane, T., Fukada, T., Muller, J. M., Shima, D. T., Hibi, M., and Hirano, T. (1999) Synergistic roles for Pim-1 and c-Myc in STAT3-mediated cell cycle progression and anti-apoptosis. Immunity 11: 709-719.

Silverman, E. S., and Collins, T. (1999) Pathways of Egr-1-mediated gene transcription in vascular biology. Am. J. Pathol. 154: 665-670.

St. Croix, B., Rago, C., Velculescu, V., Traverso, G., Romans, K. E., Montgomery, E., Lal, A., Riggins, G. J., Lengauer, C., Vogelstein, B., Kinzler, K. W. (2000) Genes expressed in human tumor endothelium. Science 289: 1197-1202.

Swanson, J. A., Johnson, M. T., Beningo, K., Post, P., Mosseker, M. and Araki, N. (1999) A contractile activity that closes phagosomes in macrophages. J. Cell Sci. 112: 307-316.

Takashima, S., and Klagsbrun, M. (1996) Inhibition of endothelial cell growth by macrophage-like U-937 cell-derived oncostatin M, leukemia inhibitory factor, and transforming growth factor beta 1. J. Biol. Chem. 271: 24901-24906.

Tye, B. K. (1999) MCM proteins in DNA replication. Ann. Rev. Biochem. 68: 649-686.

Uyttendaele, H., Closson, V., Wu, G., Roux, F., Weinmaster, G., and Kitajewski, J. (2000) Notch4 and jagged-1 induce microvessel differentiation of rat brain endothelial cells. Microvasc. Res. 60: 91-103.

Uzawa, K., Grzesik, W. J., Nishiura, T., Kuznetsov, S. A., Robey, P. G., Bren, D. A., Yamauchi, M. (1999). Differential expression of human lysyl hydroxylase genes, lysine hydroxylation, and cross-linking of type 1 collagen during osteoblastic differentiation in vitro. J. Bone Miner. Res. 14: 1272-1280.

Velculescu, V. E., Zhang, L., Vogelstein, B., and Kinzler, K. W. (1995) Serial analysis of gene expression. Science 270: 484-487.

Vernon, R. B. and Sage, E. H. (1995) Between molecules and morphology: Extracellular matrix and creation of vascular form. Am. J. Pathol. 147: 873-883.

Vernon, R. B.; Sage, E. H. A novel, quantitative model for study of endothelial cell migration and sprout formation within three-dimensional collagen matrices. Microvasc. Res. 57: 118-133; 1999.

Vezza, R., Habib, A., and FitzGerald, G. A. (1999) Differential signaling by the thromboxane receptor isoforms via the novel GTP-binding protein, Gh. J. Biol. Chem. 274: 12774-12779.

Vistin, R., Craig, K., Hwang, E. S., Prinz, S., Tyers, M., and Amon, A. (1998). The Phosphatase Cdc 14 Triggers Mitotic Exit by Reversal of Cdk-Dependent Phosphorylation. Molecular Cell. 2: 709-718.

Wahl, G. M., and Berger, S. L. (1987) Screening colonies or plaques with radioactive nucleic acid probes. Meth. Enzymol. 152: 415-423.

Wong, E. S., Lim, J., Low, B. C., Chen, Q., and Guy, C. R. (2000) Evidence for direct interaction between sprouty and cbl. J. Biol. Chem. 276: 5866-5875.

Yancopoulos, G. D., Davis, S., Gale, N. W., Rudge, J. S., Wiegand, S. J. and Holash, J. (2000) Vascular-specific growth factors and blood vessel formation. Nature 407: 242-248.

Yang, S., Graham, J., Kahn, J. W., Schwartz, E. A., and Gerritsen, M. E. (1999) Functional roles for PECAM-1 (CD31) and VE-cadherin (CD 144) in tube assembly and lumen formation in three-dimensional collagen gels. Am. J. Pathol. 155: 887-895.

Zhang, J., Tucholski, J., Lesort, M., Jope, R. S., and Johnson, G. V. (1999) Novel bimodal effects of the G-protein tissue transglutaminase on adrenoreceptor signalling. Biochem. J. 343: 541-549.

Zhang, K. Z., Westberg, J. A., von Boguslawsky, K., Lindsberg, P., Erlander, M., Guo, H., Su, J., Andersson, L. C. (1998). High expression of stanniocalcin in differentiated brain neurons. Am. J. Pathology. 153: 439-445.

Zhang, K. Z., Lindsberg, P. J., Tatlisumak, T., Kaste, M., Olsen, H. S., and Andersson, L. C. (2000) Stanniocalcin: A molecular guard of neurons during cerebral ischemia. Proc. Natl. Acad. Sci. USA 97: 3637-3642.

Zhang, X., Wrzeszczynska, M. H., Horvath, C. M., and Darnell, J. E. (1999) Interacting regions in Stat3 and c-jun that participate in cooperative transcriptional activation. Mol. Cell Biol. 19: 7138-7146.

Zimrin, A. B., Pepper, M. S., McMahon, G. A., Nguyen, F., Montesano, R., and Maciag, T. (1996) An antisense oligonucleotide to the notch ligand jagged enhances fibroblast growth factor-induced angiogenesis in vitro. J. Biol. Chem. 271: 32499-32502.

What is claimed is:

1. An isolated nucleic acid molecule segment comprising a structural nucleic acid sequence selected from the group consisting of:
a nucleic acid sequence at least about 90% identical to SEQ ID NO:1; and
a nucleic acid sequence that hybridizes under stringent hybridization conditions to the reverse complement of SEQ ID NO:1.

2. The nucleic acid molecule segment of claim 1, wherein the structural nucleic acid sequence is SEQ ID NO:1.

3. An isolated nucleic acid molecule segment comprising a structural nucleic acid sequence which encodes:
an amino acid sequence at least about 90% identical to SEQ ID NO:2; and
an amino acid sequence immunoreactive with an antibody prepared using SEQ ID NO:2 as an antigen, the antibody being immunoreactive with SEQ ID NO:2.

4. The isolated nucleic acid molecule segment of claim 3, wherein the structural nucleic acid sequence encodes SEQ ID NO:2.

5. A recombinant vector comprising operatively linked in the 5′ to 3′ orientation:
a promoter that directs transcription of a structural nucleic acid sequence;
a structural nucleic acid sequence selected from the group consisting of:
a nucleic acid sequence at least about 90% identical to SEQ ID NO:1;
a nucleic acid sequence that hybridizes under stringent hybridization conditions to the reverse complement of SEQ ID NO:1;
a nucleic acid sequence which encodes an amino acid sequence at least about 90% identical to SEQ ID NO:2; and
a nucleic acid sequence which encodes an amino acid sequence immunoreactive with an antibody prepared using SEQ ID NO:2 as an antigen, the antibody being immunoreactive with SEQ ID NO:2;
a 3′ transcription terminator.

6. The recombinant vector of claim 5, wherein the structural nucleic acid sequence is SEQ ID NO:1.

7. The recombinant vector of claim 5, wherein the structural nucleic acid sequence encodes SEQ ID NO:2.

8. A recombinant host cell comprising a structural nucleic acid sequence selected from the group consisting of:
a nucleic acid sequence at least about 90% identical to SEQ ID NO:1; and
a nucleic acid sequence that hybridizes under stringent hybridization conditions to the reverse complement of SEQ ID NO:11;
a nucleic acid sequence which encodes an amino acid sequence at least about 90% identical to SEQ ID NO:2; and
a nucleic acid sequence which encodes an amino acid sequence immunoreactive with an antibody prepared using SEQ ID NO:2 as an antigen, the antibody being immunoreactive with SEQ ID NO:2;
wherein the copy number of the structural nucleic acid sequence in the recombinant host cell is higher than the copy number of the structural nucleic acid sequence in a wild type host cell of the same species.

9. The recombinant host cell of claim 8, wherein the structural nucleic acid sequence is SEQ ID NO:1.

10. The recombinant host cell of claim 8, wherein the structural nucleic acid sequence encodes SEQ ID NO:2.

11. The recombinant host cell of claim 8, wherein the copy number of the structural nucleic acid sequence in the wild type host cell is zero.

12. The recombinant host cell of claim 8, wherein the recombinant host cell is a bacterial cell.

13. The recombinant host cell of claim 8, wherein the recombinant host cell is an Escherichia coli cell.

14. The recombinant host cell of claim 8, wherein the recombinant host cell is a fungal cell.

15. The recombinant host cell of claim 8, wherein the recombinant host cell is an insect cell.

16. An isolated protein comprising an amino acid sequence selected from the group consisting of:
an amino acid sequence at least about 90% identical to SEQ ID NO:2; and
an amino acid sequence immunoreactive with an antibody prepared using SEQ ID NO:2 as an antigen, the antibody being immunoreactive with SEQ ID NO:2.

17. The isolated protein of claim 16, wherein the amino acid sequence is SEQ ID NO:2.

18. An antibody prepared using SEQ ID NO:2 as an antigen, wherein the antibody is immunoreactive with SEQ ID NO:2.

19. A method of preparing a recombinant host cell, the method comprising:
selecting a host cell;
transforming the host cell with a recombinant vector; and
obtaining recombinant host cells; wherein the recombinant vector comprises a structural nucleic acid sequence selected from the group consisting of:
a nucleic acid sequence at least about 90% identical to SEQ ID NO:1;
a nucleic acid sequence that hybridizes under stringent hybridization conditions to the reverse complement of SEQ ID NO:1;
a nucleic acid sequence which encodes an amino acid sequence at least about 90% identical to SEQ ID NO:2; and
a nucleic acid sequence which encodes an amino acid sequence immunoreactive with an antibody prepared using SEQ ID NO:2 as an antigen, the antibody being immunoreactive with SEQ ID NO:2.

20. The method of claim 19, wherein the structural nucleic acid sequence is SEQ ID NO:1.

21. The method of claim 19, wherein the structural nucleic acid sequence encodes SEQ ID NO:2.

22. The method of claim 19, wherein the host cell is a bacterial cell.

23. The method of claim 19, wherein the host cell is an Escherichia coli cell.

24. The method of claim 19, wherein the host cell is a fungal cell.

25. The method of claim 19, wherein the host cell is an insect cell.

26. An isolated nucleic acid molecule segment comprising a structural nucleic acid sequence selected from the group consisting of:
a nucleic acid sequence at least about 90% identical to SEQ ID NO:3; and
a nucleic acid sequence that hybridizes under stringent hybridization conditions to the reverse complement of SEQ ID NO:3.

27. The nucleic acid molecule segment of claim 26, wherein the structural nucleic acid sequence is SEQ ID NO:3.

28. An isolated nucleic acid molecule segment comprising a structural nucleic acid sequence which encodes:
an amino acid sequence at least about 90% identical to SEQ ID NO:4; and
an amino acid sequence immunoreactive with an antibody prepared using SEQ ID NO:4 as an antigen, the antibody being immunoreactive with SEQ ID NO:4.

29. The isolated nucleic acid molecule segment of claim 28, wherein the structural nucleic acid sequence encodes SEQ ID NO:4.

30. A recombinant vector comprising operatively linked in the 5′ to 3′ orientation:
a promoter that directs transcription of a structural nucleic acid sequence;
a structural nucleic acid sequence selected from the group consisting of:
a nucleic acid sequence at least about 90% identical to SEQ ID NO:3;
a nucleic acid sequence that hybridizes under stringent hybridization conditions to the reverse complement of SEQ ID NO:3;
a nucleic acid sequence which encodes an amino acid sequence at least about 90% identical to SEQ ID NO:4; and
a nucleic acid sequence which encodes an amino acid sequence immunoreactive with an antibody prepared using SEQ ID NO:4 as an antigen, the antibody being immunoreactive with SEQ ID NO:4;
a 3′ transcription terminator.

31. The recombinant vector of claim 30, wherein the structural nucleic acid sequence is SEQ ID NO:3.

32. The recombinant vector of claim 30, wherein the structural nucleic acid sequence encodes SEQ ID NO:4.

33. A recombinant host cell comprising a structural nucleic acid sequence selected from the group consisting of:
a nucleic acid sequence at least about 90% identical to SEQ ID NO:3; and
a nucleic acid sequence that hybridizes under stringent hybridization conditions to the reverse complement of SEQ ID NO:3;
a nucleic acid sequence which encodes an amino acid sequence at least about 90% identical to SEQ ID NO:4; and
a nucleic acid sequence which encodes an amino acid sequence immunoreactive with an antibody prepared using SEQ ID NO:4 as an antigen, the antibody being immunoreactive with SEQ ID NO:4;
wherein the copy number of the structural nucleic acid sequence in the recombinant host cell is higher than the copy number of the structural nucleic acid sequence in a wild type host cell of the same species.

34. The recombinant host cell of claim 33, wherein the structural nucleic acid sequence is SEQ ID NO:3.

35. The recombinant host cell of claim 33, wherein the structural nucleic acid sequence encodes SEQ ID NO:4.

36. The recombinant host cell of claim 33, wherein the copy number of the structural nucleic acid sequence in the wild type host cell is zero.

37. The recombinant host cell of claim 33, wherein the recombinant host cell is a bacterial cell.

38. The recombinant host cell of claim 33, wherein the recombinant host cell is an Escherichia coli cell.

39. The recombinant host cell of claim 33, wherein the recombinant host cell is a fungal cell.

40. The recombinant host cell of claim 33, wherein the recombinant host cell is an insect cell.

41. An isolated protein comprising an amino acid sequence selected from the group consisting of:
an amino acid sequence at least about 90% identical to SEQ ID NO:4; and
an amino acid sequence immunoreactive with an antibody prepared using SEQ ID NO:4 as an antigen, the antibody being immunoreactive with SEQ ID NO:4.

42. The isolated protein of claim 41, wherein the amino acid sequence is SEQ ID NO:4.

43. An antibody prepared using SEQ ID NO:4 as an antigen, wherein the antibody is immunoreactive with SEQ ID NO:4.

44. A method of preparing a recombinant host cell, the method comprising:
selecting a host cell;
transforming the host cell with a recombinant vector; and
obtaining recombinant host cells; wherein the recombinant vector comprises a structural nucleic acid sequence selected from the group consisting of:
a nucleic acid sequence at least about 90% identical to SEQ ID NO:3;
a nucleic acid sequence that hybridizes under stringent hybridization conditions to the reverse complement of SEQ ID NO:3;
a nucleic acid sequence which encodes an amino acid sequence at least about 90% identical to SEQ ID NO:4; and
a nucleic acid sequence which encodes an amino acid sequence immunoreactive with an antibody prepared using SEQ ID NO:4 as an antigen, the antibody being immunoreactive with SEQ ID NO:4.

45. The method of claim 44, wherein the structural nucleic acid sequence is SEQ ID NO:3.

46. The method of claim 44, wherein the structural nucleic acid sequence encodes SEQ ID NO:4.

47. The method of claim 44, wherein the host cell is a bacterial cell.

48. The method of claim 44, wherein the host cell is an Escherichia coli cell.

49. The method of claim 44, wherein the host cell is a fungal cell.

50. The method of claim 44, wherein the host cell is an insect cell.

51. An isolated fusion protein, comprising:
a first amino acid sequence at least about 90% identical to SEQ ID NO:5; and
a second amino acid sequence.

52. The fusion protein of claim 51, wherein the first amino acid sequence is SEQ ID NO:4.

53. The fusion protein of claim 51, wherein the first amino acid sequence is SEQ ID NO:5.

54. The fusion protein of claim 51, wherein the second amino acid sequence is a polyhistidine tag sequence.

55. The fusion protein of claim 51, wherein the second amino acid sequence is a green fluorescent protein sequence.

56. A method of isolating a matrix protein, the method comprising:
contacting the matrix protein and a CMG protein to produce a matrix protein—CMG protein complex;
isolating the matrix protein—CMG protein complex; and
dissociating the matrix protein—CMG protein complex;
obtaining an isolated the matrix protein; wherein the CMG protein comprises an amino acid sequence selected from the group consisting of:
an amino acid sequence at least about 90% identical to SEQ ID NO:4; and
an amino acid sequence immunoreactive with an antibody prepared using SEQ ID NO:4 as an antigen, the antibody being immunoreactive with SEQ ID NO:4.

57. The method of claim 56, wherein the amino acid sequence is SEQ ID NO:4.

58. The method of claim 56, wherein the matrix protein is collagen type IV or laminin.

59. A method of regulating angiogenesis, the method comprising contacting cells with an viral vector encoding a CMG-2 protein or a CMG-2 protein fragment.

60. The method of claim 59, wherein the cells are mammalian cells.

61. The method of claim 59, wherein the contacting step is performed in vivo.

62. The method of claim 59, wherein the CMG-2 protein is SEQ ID NO:4.

63. The method of claim 59, wherein the viral vector is an adenoviral vector.

64. The method of claim 59, wherein the viral vector comprises SEQ ID NO:3.

65. A method of regulating angiogenesis, the method comprising contacting cells with an antisense nucleic acid molecule, wherein the antisense nucleic acid molecule hybridizes to mRNA encoding a CMG-2 protein.

66. The method of claim 65, wherein the mRNA is transcribed from a nucleic acid sequence comprising SEQ ID NO:3.

67. The method of claim 65, wherein the CMG-2 protein is SEQ ID NO:4.

68. The method of claim 65, further comprising contacting the cells with a viral vector encoding the antisense nucleic acid molecule.


SEQUENCE LISTING (21 sequences)

SEQ ID NO: 1 - DNA, 1800 bp, Homo sapiens
atggccagcaatcacaaatcttcagcagctcgccctgtttcaagaggtggagttgggttaacaggaaggcctccttctgggatacgacccctatcaggaaatattcgagtggcaactgcaatgccacctgggacagcaagaccaggttctcgtggttgtcccatagggactggtggagttctgtcttctcaaatcaaagttgcccatcgccctgtaacacaacaaggtttgactggaatgaaaactgggacgaaaggtccccagaggcaaattttagacaaatcttactatcttgggcttcttagaagtaaaataagtgaacttacaactgaagttaataaacttcagaagggaatagaaatgtacaatcaagagaattcagtatatttgtcatatgaaaagagggctgagactttagctgttgagataaaagagcttcaaggacaactagcagactacaacatgttggtagataaacttaataccaacactgaaatggaagaagtaatgaatgattacaatatgcttaaagctcaaaatgatcgagaaacacaaagtttggatgtcatatttactgaaagacaagcgaaagaaaaacaaatcagaagtgtcgaagaagaaattgaacaggaaaaacaagcaacagatgacattatcaaaaatatgtcttttgaaaaccaagtcaagtacctagagatgaaaaccacaaatgagaaactgttacaggaattagatacacttcaacaacaattggattcacagaacatgaaaaaagagagcctggaagcagaaatagctcactcccaggtgaaacaggaggcggtattgctgcatgaaaaactttatgagttggagtcccatcgagatcaaatgattgcagaagacaaaagcataggatctccaatggaagagagagagaaattacttaagcagattaaagatgataatcaggaaatagccagcatggaaagacagttaacagatacaaaagaaaagataaatcagtttattgaagaaattagacaacttgacatggatttagaggaacaccaaggtgaaatgaaccagaaatacaaggagctaaagaaaagggaggaacatatggacacttttattgagacttttgaggaaacaaagaatcaggaactgaaacgaaaggcacagatagaagccaacattgttgcactcttggagcactgcagtcgaaatataaatcgtatagaacagatatcctctatcaccaatcaagagctaaagatgatgcaggatgacctcaattttaaatctactgaagtgcagaaatcacaaagtacagctcagaatttgacttcagacattcaacgtctgcagttggatctgcagaaaatggagcttctagaaagtaagatgactgaagaacagcattctctaaaaagcaaaattaagcaaatgacaactgatctggagatatataatgatttgccagctttaaaatcatcaggtgaagaaaagataaagaaattacatcaggagagaatgatattatcaacccacagaaatgcctttaagaaaataatggagaagcaaaacatagagtatgaggcactaaaaacacaattgcaagaaaatgagacacattctcagcttacaaatttggagagaaagtggcaacaccttgagcaaaataattttgcgatgaaagaattcatagcaaccaagagtcaagagagtgattaccagccaattaagaaaaatgtgaccaagcagattgcagagtacaataaaaccatcgtggatgctttacatagcaccagcggaaac

SEQ ID NO: 2 - protein, 600 aa, Homo sapiens
MASNHKSSAARPVSRGGVGLTGRPPSGIRPLSGNIRVATAMPPGTARPGSRGCPIGTGGVLSSQIKVAHRPVTQQGLTGMKTGTKGPQRQILDKSYYLGLLRSKISELTTEVNKLQKGIEMYNQENSVYLSYEKRAETLAVEIKELQGQLADYNMLVDKLNTNTEMEEVMNDYNMLKAQNDRETQSLDVIFTERQAKEKQIRSVEEEIEQEKQATDDIIKNMSFENQVKYLEMKTTNEKLLQELDTLQQQLDSQNMKKESLEAEIAHSQVKQEAVLLHEKLYELESHRDQMIAEDKSIGSPMEEREKLLKQIKDDNQEIASMERQLTDTKEKINQFIEEIRQLDMDLEEHQGEMNQKYKELKKREEHMDTFIETFEETKNQELKRKAQIEANIVALLEHCSRNINRIEQISSITNQELKMMQDDLNFKSTEVQKSQSTAQNLTSDIQRLQLDLQKMELLESKMTEEQHSLKSKIKQMTTDLEIYNDLPALKSSGEEKIKKLHQERMILSTHRNAFKKIMEKQNIEYEALKTQLQENETHSQLTNLERKWQHLEQNNFAMKEFIATKSQESDYQPIKKNVTKQIAEYNKTIVDALHSTSGN

SEQ ID NO: 3 - DNA, 1158 bp, Homo sapiens
atggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaagtctgggagtgtggcaaataactggattgaaatttataatttcgtacagcaacttgcggagagatttgtgagccctgaaatgagattatctttcattgtgttttcttctcaagcaactattattttgccattaactggagacagaggcaaaatcagtaaaggcttggaggatttaaaacgtgttagtccagtaggagagacatatatccatgaaggactaaagctagcgaatgaacaaattcagaaagcaggaggcttgaaaacctccagtatcataattgctctgacagatggcaagttggacggtctggtgccatcatatgcagagaaagaggcaaagatatccaggtcacttggggctagtgtttattgtgttggtgtccttgattttgaacaagcacagcttgaaagaattgctgattccaaggagcaagttttccctgtcaaaggtggatttcaggctcttaaaggaataattaattcttctaacgggatcgcagccatcattgttattttggtgttactgctactcctggggatcggtttgatgtggtggttttggcccctttgctgcaaagtggttattaaggatcctccaccaccacccccccctgcaccaaaagaggaggaagaagaacctttgcctactaaaaagtggccaactgtggatgcttcctattatggtggtcgaggggttggaggaattaaaagaatggaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaagggtcgtcttgatgctctctgggctttgttgaggcggcagtatgaccgggtttctttgatgcgacctcaggaaggagatgaggtttgtatatgggaatgtattgagaaagagctaactgct

SEQ ID NO: 4 - protein, 386 aa, Homo sapiens [sequence omitted]

SEQ ID NO: 5 - protein, 181 aa, Homo sapiens
QEQPSCRRAFDLYFVLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSSN

SEQ ID NO: 6 - DNA, 26 bp, Artificial sequence
  3′ end is from Homo sapiens; 5′ end is engineered to add restriction site
agctcgagacaatggccagcaatcac

SEQ ID NO: 7 - DNA, 27 bp, Artificial sequence
  3′ end is from Homo sapiens; 5′ end is engineered to add restrict ion site
agggatccggtttccgctggtgctatg

SEQ ID NO: 8 - DNA, 27 bp, Artificial sequence
  3′ end is from Homo sapiens; 5′ end is engineered to add restriction site
agctcgagaggatggtggcggagcggt

SEQ ID NO: 9 - DNA, 26 bp, Artificial sequence
  3′ end is from Homo sapiens; 5′ end is engineered to add restriction site
aggaattcagcagttagctctttctc

SEQ ID NO: 10 - DNA, 28 bp, Artificial sequence
  3′ end is from Homo sapiens; 5′ end is engineered to add restriction site
agtctagattatgatctagagtcgcggc

SEQ ID NO: 11 - DNA, 26 bp, Artificial sequence
  3′ end is from Homo sapiens; 5′ end is engineered to add restriction site
agagatctacaatggccagcaatcac

SEQ ID NO: 12 - DNA, 27 bp, Artificial sequence
  3′ end is from Homo sapiens; 5′ end is engineered to add restriction site
agctcgagaggatggtggcggagcggt

SEQ ID NO: 13 - DNA, 26 bp, Artificial sequence
  3′ end is from Homo sapiens; 5′ end is engineered to add restriction site
agggatcccaggagcagccctcctgc

SEQ ID NO: 14 - DNA, 26 bp, Artificial sequence
  3′ end is from Homo sapiens; 5′ end is engineered to add restriction site
agaagcttagaagaattaattattcc

SEQ ID NO: 15 - DNA, 1958 bp, Homo sapiens
ctgagcgtgggcctcagaaagaagttaaggcacccgcgagccgggcaactgccctccttccgcgccggcggagcgattaaagtgaagaaacaatggccagcaatcacaaatcttcagcagctcgccctgtttcaagaggtggagttgggttaacaggaaggcctccttctgggatacgacccctatcaggaaatattcgagtggcaactgcaatgccacctgggacagcaagaccaggttctcgtggttgtcccatagggactggtggagttctgtcttctcaaatcaaagttgcccatcgccctgtaacacaacaaggtttgactggaatgaaaactgggacgaaaggtccccagaggcaaattttagacaaatcttactatcttgggcttcttagaagtaaaataagtgaacttacaactgaagttaataaacttcagaagggaatagaaatgtacaatcaagagaattcagtatatttgtcatatgaaaagagggctgagactttagctgttgagataaaagagcttcaaggacaactagcagactacaacatgttggtagataaacttaataccaacactgaaatggaagaagtaatgaatgattacaatatgcttaaagctcaaaatgatcgagaaacacaaagtttggatgtcatatttactgaaagacaagcgaaagaaaaacaaatcagaagtgtcgaagaagaaattgaacaggaaaaacaagcaacagatgacattatcaaaaatatgtcttttgaaaaccaagtcaagtacctagagatgaaaaccacaaatgagaaactgttacaggaattagatacacttcaacaacaattggattcacagaacatgaaaaaagagagcctggaagcagaaatagctcactcccaggtgaaacaggaggcggtattgctgcatgaaaaactttatgagttggagtcccatcgagatcaaatgattgcagaagacaaaagcataggatctccaatggaagagagagagaaattacttaagcagattaaagatgataatcaggaaatagccagcatggaaagacagttaacagatacaaaagaaaagataaatcagtttattgaagaaattagacaacttgacatggatttagaggaacaccaaggtgaaatgaaccagaaatacaaggagctaaagaaaagggaggaacatatggacacttttattgagacttttgaggaaacaaagaatcaggaactgaaacgaaaggcacagatagaagccaacattgttgcactcttggagcactgcagtcgaaatataaatcgtatagaacagatatcctctatcaccaatcaagagctaaagatgatgcaggatgacctcaattttaaatctactgaagtgcagaaatcacaaagtacagctcagaatttgacttcagacattcaacgtctgcagttggatctgcagaaaatggagcttctagaaagtaagatgactgaagaacagcattctctaaaaagcaaaattaagcaaatgacaactgatctggagatatataatgatttgccagctttaaaatcatcaggtgaagaaaagataaagaaattacatcaggagagaatgatattatcaacccacagaaatgcctttaagaaaataatggagaagcaaaacatagagtatgaggcactaaaaacacaattgcaagaaaatgagacacattctcagcttacaaatttggagagaaagtggcaacaccttgagcaaaataattttgcgatgaaagaattcatagcaaccaagagtcaagagagtgattaccagccaattaagaaaaatgtgaccaagcagattgcagagtacaataaaaccatcgtggatgctttacatagcaccagcggaaactgagtttaagtccactgaaagtctctaaggaagtatcctcttgctgctaaacttggtacaagttga

SEQ ID NO: 16 - DNA, 1343 bp, Homo sapiens
cgctgccgcaggcgccggcgtctcagctgctcgccgccccccaccccagagtgcgtgcagggtgactcccgccacctttgcgaccctcctgagcttaggggactgcgagcgggagggagtctcaggcccccggccgcaggatggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaagtctgggagtgtggcaaataactggattgaaatttataatttcgtacagcaacttgcggagagatttgtgagccctgaaatgagattatctttcattgtgttttcttctcaagcaactattattttgccattaactggagacagaggcaaaatcagtaaaggcttggaggatttaaaacgtgttagtccagtaggagagacatatatccatgaaggactaaagctagcgaatgaacaaattcagaaagcaggaggcttgaaaacctccagtatcataattgctctgacagatggcaagttggacggtctggtgccatcatatgcagagaaagaggcaaagatatccaggtcacttggggctagtgtttattgtgttggtgtccttgattttgaacaagcacagcttgaaagaattgctgattccaaggagcaagttttccctgtcaaaggtggatttcaggctcttaaaggaataattaattcttctaacgggatcgcagccatcattgttattttggtgttactgctactcctggggatcggtttgatgtggtggttttggcccctttgctgcaaagtggttattaaggatcctccaccaccacccccccctgcaccaaaagaggaggaagaagaacctttgcctactaaaaagtggccaactgtggatgcttcctattatggtggtcgaggggttggaggaattaaaagaatggaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaagggtcgtcttgatgctctctgggctttgttgaggcggcagtatgaccgggtttctttgatgcgacctcaggaaggagatgaggtttgtatatgggaatgtattgagaaagagctaactgcttgagtcagtataatggaggcagggaaatagtaataaaaaatgatt

SEQ ID NO: 17 - protein, 33 aa, Homo sapiens
MVAERSPARSPGSWLFPGLWLLVLSGPGGLLRA

SEQ ID NO: 18 - protein, 174 aa, Homo sapiens
QEQPSCRRAFDLYFVLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALK

SEQ ID NO: 19 - protein, 9 aa, Homo sapiens
IIIALTDGK

SEQ ID NO: 20 - protein, 17 aa, Homo sapiens
GIAAIIVILVLLLLLGI

SEQ ID NO: 21 - protein, 10 aa, Homo sapiens
PPPPPPAPKE